(19) United States
(12) Patent Application Publication
(10) Pub. No.: US 2026/0102758 A1
(43) Pub. Date: Apr. 16, 2026

(54) 3D STRUCTURE-FUNCTIONAL DESIGN OF BIOMASS-DERIVED SOLAR PHOTOCATALYST FOR ANTIMICROBIAL EFFICACY AND CHEMICAL DEGRADATION AT AMBIENT CONDITIONS

(71) Applicant: THE TEXAS A&M UNIVERSITY SYSTEM, COLLEGE STATION, TX (US)

(72) Inventors: YUAN DAI, COLLEGE STATION, TX (US); WAN ZHANG, COLLEGE STATION, TX (US)

(21) Appl. No.: 19/358,770

(22) Filed: Oct. 15, 2025

Related U.S. Application Data
(60) Provisional application No. 63/707,352, filed on Oct. 15, 2024.

(51) Int. Cl. B01J 21/18; A61L 2/08; B01J 35/27; B01J 35/39; B01J 35/40; B01J 35/52; B01J 35/59; C02F 1/30; C02F 1/72; C02F 101/34; C02F 101/38

(52) U.S. Cl. CPC B01J 21/18; A61L 2/084; A61L 2/088; B01J 35/27; B01J 35/39; B01J 35/40; B01J 35/52; B01J 35/59; C02F 1/30; C02F 1/725; C02F 2101/34; C02F 2101/38; C02F 2305/08; C02F 2305/10

(57) ABSTRACT

The subject invention pertains a stable, three-dimensional (3D) photocatalyst composition comprising TiO₂ doped with carbon derived from lignin. The photocatalyst composition has a stable, hollow, spherical or spheroid nanoparticle structure, which improves photodegradation under visible light irradiation over unmodified TiO₂ photocatalysts. The subject invention also provides methods of preparing the photocatalyst, as well as methods using the photocatalyst to remediate contaminants such as pharmaceuticals, pathogens and persistent organic pollutants.

### CROSS-REFERENCE TO RELATED APPLICATION

This application claims the benefit of U.S. Provisional Application Ser. No. 63/707,352, filed Oct. 15, 2024, the disclosure of which is hereby incorporated by reference in its entirety, including all figures, tables and amino acid or nucleic acid sequences.

### STATEMENT REGARDING FEDERALLY SPONSORED RESEARCH

This invention was made with government support under R01ES032708 awarded by the National Institute of Environmental Health Sciences. The government has certain rights in the invention.

### BACKGROUND OF THE INVENTION

Over the past several decades, worldwide production and utilization of synthetic and naturally occurring chemical substances, such as, for example, pharmaceuticals, pesticides, dyes, solvents, surfactants and persistent organic substances, has led to the introduction of significant quantities of hazardous materials into the environment.

As an important example, perfluoroalkyl and polyfluoroalkyl substances (PFAS) are found widely in products such as personal care products, food packaging, pesticides and coatings, to name a few. Sometimes referred to as “forever chemicals,” PFAS are pervasive in the environment and are highly recalcitrant to degradation. There are over 9,000 alternative PFAS molecules that have been connected to conditions affecting human and wildlife health such as, for example, cancer, birth defects and infertility.

In addition, pharmaceutical compounds have been detected in ground and surface water, mainly deriving from human and veterinary medicines, such as antibiotics, antidepressants, blood thinners, hormones, and painkillers. In some instances, unused or expired medications enter waterways as a result of improper disposal, for example, when they are flushed down toilets or drains. While there have been certain efforts to prevent improper disposal of pharmaceuticals, such as drug take-back programs and disposal guidelines from the U.S. Environmental Protection Agency (EPA), these efforts do not effectively capture all drugs that might end up in the environment. This is, in part, because pharmaceuticals can also enter water systems as a result of human and animal drug consumption, given that certain concentrations of drug compounds are often released from the body unmetabolized.

Furthermore, pathogenic organisms, such as fungi, viruses and bacteria, are also ever-present environmental contaminants that pose a health risk to humans, animals and plants. Thus, soil, air, groundwater and other surfaces may contain any number of chemical or pathogenic contaminants requiring remediation. Nonetheless, most decontamination processes and sterilization techniques require harsh reaction conditions, use non-renewable resources and/or are not environmentally friendly.

In some instances, environmental contamination may be addressed via adsorption techniques, which involve the adsorption of contaminants onto a surface of a sorbent substance. Though many sorbents have been explored for environmental remediation, their use is hindered by the need for costly metals or polymer-based materials, as well as the unfortunate creation of secondary pollution. While more sustainable and lower-cost chitosan, biochar, microbial biomass and agricultural waste-based biosorbents have been studied, they lack the performance required for industrial applications.

Environmental contaminants may also be addressed via degradation, for example, microbial degradation, thermal degradation, electrochemical degradation and/or photodegradation. In particular, photodegradation, or photocatalysis, involves the application of a photocatalyst to initiate and boost chemical reactions that result in the degradation of materials in the environment into less toxic components. A photocatalyst uses incident irradiation to excite an electron from the valence band (leaving behind a hole) to the conduction band of a semiconductor. These photoinduced charge carriers then proceed to form reactive radicals, hydroxyl radicals and super oxide radicals that attack adsorbed chemicals on the surface of the material, causing them to dissociate. Semiconducting metal oxides, such as TiO₂, SnO₂, Fe₂O₃, ZnO, CeO₂, ZrO₂, WO₃ and V₂O₅, have been extensively used as photocatalysts.

Photocatalysis is gaining attention due to its mild reaction conditions, high efficiency, strong oxidation capacity, less harmful byproducts and overall low environmental impact of treated effluent. Notably, photocatalysis has the advantage of utilizing renewable solar energy; however, existing photocatalytic systems face challenges in widespread industrial implementation due to dependency on UV radiation, inefficient utilization of visible light, rapid recombination of charges and low efficiency in transferring photo-generated electrons and holes.

As such, visible light range photocatalysis would be advantageous given that contaminant removal can be achieved under ambient conditions. While metal-based visible light photocatalysts show promising catalytic activity, some are hindered by the presence of toxic elements, thus limiting their practical applications and potentially causing secondary pollution. Additionally, precious metal-based photocatalyst design creates challenges for commercialization due to the high cost.

Among various photocatalytic materials, titanium dioxide (TiO₂) is a cost-effective, photostable, avirulent and highly efficient oxide semiconductor catalyst used extensively for gas purification, sterilization and the degradation of refractory organic pollutants; however, the structural limitations of TiO₂, including very low surface area and low porosity, can result in rapid quenching of photoactive species. Additionally, it can be activated only by irradiating with ultraviolet light, which comprises only around 4-8% of the solar radiation that reaches Earth's surface, and which limits the potential for broad applications under various environmental settings. Therefore, there is a need for addressing the challenges associated with the use of TiO₂ in photocatalysis with sustainable, efficient and cost-effective solutions.

### BRIEF SUMMARY OF THE INVENTION

The subject invention provides efficient, economical and environmentally-friendly compositions and methods for remediating a contaminant from, for example, soil, air, water and other surfaces. More specifically, the subject invention provides a photocatalyst composition capable of functioning under visible light irradiation, as well as methods of using the composition to remediate a variety of contaminants, including, for example, pathogenic agents, pharmaceuticals and persistent organic pollutants.

In certain embodiments, the subject invention provides a stable, three-dimensional (3D) photocatalyst composition comprising a metal oxide doped with carbon from a carbon precursor material. In preferred embodiments, the carbon precursor material is lignin and the metal oxide it TiO₂.

In a specific embodiment, the photocatalyst composition comprises a hollow, spherical or spheroid nanoparticle structure, which is formed by establishing stable Ti—O—C and Ti—O—Ti bonds through the crosslinking of lignin hydroxyl surface functional groups with hydrolyzed Ti(OH)₄. FIG. 1A.

Advantageously, the photocatalyst composition can contribute to significant photodegradation activity under a broad range of electromagnetic radiation wavelengths (including visible light wavelengths), compared to known metal-based photocatalysts that operate only under ultraviolet (UV) wavelengths.

In certain embodiments, the photocatalyst structure is formed by a lattice of bonds that allow for uniform and stable carbon modification of TiO₂, which can improve the resistance of the photocatalyst to photocorrosion and extend the lifespan of the photocatalyst compared with unmodified TiO₂. For example, in certain embodiments, the durable lattice structural design allows for stable photocatalyst performance under visible light irradiation that retains function for at least 5 cycles of usage, or preferably at least 10 cycles of usage. Accordingly, in some embodiments, the compositions of the subject invention can be reusable and/or recyclable.

Furthermore, in certain embodiments, the nanoparticle shell structure of the photocatalyst can improve photo-utilization, including from visible light spectrum wavelengths, compared with unmodified TiO₂. The photogenerated charge is stabilized due to efficient scattering of light throughout the hollow spherical cavity, which provides ample active sites relative to surface area.

The photocatalyst composition of the subject invention can be incorporated into a variety of formulations depending upon the desired usage. For example, in some embodiments, the photocatalyst composition is formulated as part of an aqueous and/or aerosol formulation; imbued into a substrate, such as a cleaning wipe, a matrix, a membrane or a filter; and/or affixed to a surface in the form of a film or coating.

Also provided herein are methods for producing a photocatalyst according to the subject invention, comprising mixing titanium (IV) n-butoxide with a dispersion of lignin nanoparticles; copolymerizing the titanium (IV) n-butoxide with the lignin nanoparticles via hydrolysis of the titanium (IV) n-butoxide; and calcining the resulting copolymer. FIG. 1A.

The subject invention further provides methods for remediating a contaminant from a site using a photocatalyst composition according to the subject invention. In certain embodiments, the methods comprise contacting an effective amount of the photocatalyst composition with the site and irradiating the photocatalyst with electromagnetic radiation in order to initiate and/or enhance remediation of the contaminant.

The methods of the subject invention can be useful for remediating at least 50% of a pathogenic organism, pharmaceutical, persistent organic chemical or other contaminant present at a site within a time period of 12 hours, preferably within 3 hours, or less, of application of the photocatalyst in the presence of electromagnetic radiation. In certain embodiments, the method remediates a contaminant to a concentration that meets local, federal or other mandated levels. The contaminated site can be, for example, soil, air, water and/or any number of other porous or non-porous surfaces.

Advantageously, the lignin-based photocatalyst presents a sustainable approach for environmental remediation under ambient conditions, with broad applications in toxin treatment and protection against pathogenic infection in everyday life.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1A depicts a schematic illustration of the C_{lignin}@H—TiO₂ synthetic procedure.

FIG. 1B depicts FTIR of Lignin@H—TiO₂, TiO₂ mixed with 5% lignin, TiO₂ and lignin. FIGS. 1C-1E depict XPS spectra for Lignin@H—TiO₂ of carbon 1s (FIG. 1C), Ti 2p (FIG. 1D), and oxygen 1s (FIG. 1E).

FIG. 2A depicts SEM morphology of the C_{lignin}@H—TiO₂ nanoparticles. FIG. 2B depicts TEM morphology of the C_{lignin}@H—TiO₂ nanoparticles. FIGS. 2C-2E depict EDS morphology of the C_{lignin}@H—TiO₂ nanoparticles: carbon (FIG. 2C); Ti (FIG. 2D); and oxygen (FIG. 2E).

FIG. 3 depicts particle size analysis of C_{lignin}@H—TiO₂ nanoparticles.

FIG. 4A depicts the lattice fringes and FIG. 4B depicts the crystal pattern of C_{lignin}@H—TiO₂.

FIG. 5A depicts Tg and FIG. 5B depicts the calculated C_{lignin} content of C_{lignin}@H—TiO₂ synthesized with different lignin weight percentages.

FIG. 6A depicts XRD patterns of C_{lignin}@H—TiO₂ and FIG. 6B depicts Atenolol degradation performance by the C_{lignin}@H—TiO₂ materials synthesized at varying temperatures.

FIGS. 7A-7F depict atenolol degradation performance of TiO₂ based samples (each point represents triplicate measurements, FIG. 7A); XRD of C_{lignin}@H—TiO₂ materials synthesized at varying temperatures (FIG. 7B); atenolol degradation with different lignin content (each point represents triplicate measurements, FIG. 7C); UV-Vis spectra of C_{lignin}@H—TiO₂ with varying lignin concentrations (FIG. 7D); the (αhν)^{1/2} versus (hv) plot of C_{lignin}@H—TiO₂ with varying lignin concentrations (FIG. 7E); and performance of the C_{lignin}@H—TiO₂ photocatalyst (10 mins radiation) upon successive 10 cycles (FIG. 7F).

FIG. 8 depicts the changes in morphology of the C_{lignin}@H—TiO₂ nanoparticle synthesized with different weight percentages of lignin.

FIG. 9 depicts electrical conductivity of C_{lignin}@TiO₂ and H—TiO₂.

FIG. 10 depicts XRD patterns of C_{lignin}@H—TiO₂ materials synthesized with different lignin content at 600° C. for 90 min.

FIG. 11A depicts the effect of quenchers on the atenolol degradation performance (each point represents triplicate measurements). FIG. 11B depicts a schematic diagram of degradation mechanism (VB: valence band; CB: conduction band).

FIG. 12 depicts DMPO spin-trapping ESR spectra of C_{lignin}@H—TiO₂ with solar light (top line tracing) and C_{lignin}@H—TiO₂ in dark (bottom line tracing).

FIG. 13A depicts electrochemical impedance of anatase TiO₂, anatase TiO₂ with solar light, C_{lignin}@H—TiO₂ and C_{lignin}@H—TiO₂ with solar light. FIG. 13B depicts photocurrent response of anatase TiO₂ and C_{lignin}@H—TiO₂. FIG. 13C depicts steady-state photoluminescence emission (PL) spectra of anatase TiO₂ and C_{lignin}@H—TiO₂ after excitation (Exc) at 350 nm.

FIG. 14A depicts fluorescence signal intensity, suggesting the bacterial oxidative stress (the stress is probed by transforming a redox-sensitive green fluorescence protein fusion (i.e., ORP-GFP) into the cell) under solar light in the presence of C_{lignin}@H—TiO₂ (percentage represents the light intensity). FIG. 14B depicts plasmid degradation at different radiation time. FIG. 14C depicts antibacterial performance of C_{lignin}@H—TiO₂ with different catalyst dosage. FIG. 14D depicts antibacterial performance of C_{lignin}@H—TiO₂ applied on protective mobile case. FIG. 14E depicts PFOA degradation by C_{lignin}@H—TiO₂ at different catalyst quantities. FIG. 14F depicts atenolol degradation using C_{lignin}@H—TiO₂ membrane over time.

FIG. 15 depicts antibacterial performance of the C_{lignin}@H—TiO₂ photocatalyst.

### DETAILED DISCLOSURE OF THE INVENTION

The subject invention provides a carbon-doped metal oxide photocatalyst composition capable of functioning under visible light irradiation, as well as methods for producing the composition and methods of using the composition in remediation of contaminants.

As used herein, each of the following terms have the meanings associated with it as specified below. Unless defined otherwise, all technical and scientific terms used herein generally have the same meaning as commonly understood by one of ordinary skill in the art to which this invention belongs.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. In the context of reagent and/or analyte concentrations, the term “about” can mean a range of up to 0-20%, 0 to 10%, 0 to 5%, or up to 1% of a given value. Thus, in the context of compositions containing amounts of ingredients where the terms “about” or “approximately” are used, these compositions contain the stated amount of the ingredient with a variation (error range) of 0-10% around the value (X±10%), of 0-5% around the value (X±5%), or up to 1% around the value (X±1%). In the context of pH measurements, the terms “about” or “approximately” permit a variation of +0.1 unit from a stated value.

In the subject disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

As used herein, “contaminant” refers to any substance that causes another substance or object to become fouled, polluted or impure. Contaminants can be living or non-living and can be inorganic or organic substances or deposits, including, but not limited to, pathogens and other undesirable microorganisms; pharmaceuticals; persistent organic pollutants (POPs); polychlorinated biphenyls (PCBs); halogenated volatile organic compounds (CVOCs), such as tetrachloroethene (PCE), trichloroethene (TCE), trichloroethane (TCA), dichloroethene (DCE), vinyl chloride; fuel constituents such as benzene, ethylbenzene, toluene, xylene, methyl tert butyl ether (MTBE), tertiary butyl alcohol (TBA), polynuclear aromatic hydrocarbons (PAHs), ethylene dibromide (EDB); pesticides such as (but not limited to) DDT; herbicides such as (but not limited to) Silvex; heavy metals; coatings, paints, inks and dyes; hydrocarbons, such as petroleum, tar or asphaltenes; lipids, fats, oils and greases, such as cooking grease, plant-based oils, and lard; waxes, such as paraffin; resins; textile fibers; quaternary ammonium compounds (QACs); plastics and polymers; aqueous film-forming foam (AFFF); and radioactive waste.

As used herein, a “pathogen” refers to any single-celled or acellular organism that is capable of causing an infection, disease or other form of harm in another organism. As used herein, pathogenic microorganisms are infectious agents and can include, for example, bacteria, cyanobacteria, biofilms, viruses, virions, viroids, fungi, molds, mildews, protozoa, prions, and algae. Non-limiting examples of bacterial pathogens include Streptococcus spp. (e.g., S. agalactiae, S. pneumoniae, S. pyogenes, S. salivarius, and S. sanguis); Staphylococcus spp. (e.g., S. aureus, S. epidermidis, S. haemolyticus, S. hominis, and S. simulans, as well as oxacillin-resistant (ORSA) and oxacillin-susceptible staphylococci (also known as methicillin-resistant [MRSA] or methicillin-susceptible staphylococci)); Acinetobacter spp. (e.g., A. baumanii); Bacillus spp. (e.g., B. anthracis, B. cereus); Bacteroides spp. (e.g., B. fragilis); Campylobacter spp.; Clostridium spp. (e.g., C. difficile); Enterobacter spp.; Enterococcus spp. (e.g., E. faecalis and E. faecium, vancomycin-susceptible and vancomycin-resistant strains); Escherichia coli; Francisella spp.; Giardiasis spp.; Helicobater spp. (e.g., H. pylori); Klebsiella spp. (e.g., K. aerogenes, K. pneumonia); Legionella spp. (e.g., L. pneumophila); Mycobacterium spp. (e.g., M. bovis, M. tuberculosis); Propionibacterium spp.; Proteus spp. (e.g., P. mirabilis); Pseudomonas spp. (e.g., P. aeruginosa); Salmonella spp. (e.g., S. enterocolitis; S. enterica, S. choleraseus); Selenomonas spp.; Shigella spp. (e.g., S. dysenteriae); Stenotrophomonas spp.; Veillonella spp.; Vibrio spp. (e.g., V. cholera, V. parahaemolytics); and Yersinia spp. (e.g., Y. pestis).

Non-limiting examples of viral pathogens include coronaviruses (including SARS-CoV1 and CoV2), rotaviruses, norovirus, hepatitis A, B, and C, Coxsackievirus, Rhinovirus, the cold virus, influenza, herpes viruses, cytomegalovirus, and poliovirus.

Non-limiting examples of fungal pathogens include Zygosaccharomyces spp., Debaryomyces spp. (e.g., D. hansenii), Candida spp. (e.g., C. albicans, C. auris), Dekkera/Brettanomyces spp., Leptosphaerulina spp. (e.g., L. chartarum), Epicoccum spp. (e.g., E. nigrum), Wallemia spp. (e.g., W. sebi), Cryptococcus spp., Trichophyton spp. (e.g., T. rubrum, T. mentagrophytes), Epidermophyton spp. (e.g., E. floccosum), and Mucor spp. (e.g., M. miehei).

As used herein, an “undesirable” microorganism refers to a non-pathogenic species whose growth on a surface or in a product can cause visible growth, odors, spoilage, or other organoleptic damage to a product. While not necessarily capable of causing an infection, such organisms can spoil foods, soil surfaces, create visible biofilms and/or create undesirable odors. Their growth can make products unfit for use, especially foods, cosmetics, cleaning products, and personal care items. Non-limiting examples of undesirable microorganisms that cause spoilage or other physical but nonpathogenic changes to a product include certain species within the bacterial genera Lactobacillus, Pediococcus, Micrococcus, Streptococci, Propionibacterium, Streptomyces, Actinomycetes, and Bacilli, and fungal genera including Geotrichum, Penicillium, Saccharomyces, and Zygosaccharomyces.

As used herein, “persistent organic pollutants” or “POPs” refer to organic compounds that are resistant to degradation through chemical, biological and photolytic processes. POPs can include, for example, pesticides, solvents, pharmaceuticals and industrial chemicals. Specific non-limiting examples include aldrin, chlordane, dieldrin, endrin, heptachlor, HCB, mirex, toxaphene, PCBs, DDT, dioxins, polychlorinated dibenzofurans and perfluoroalkyl and polyfluoroalkyl substances (PFAS). PFAS can include, for example, perfluorooctane sulfonate (PFOS); perfluorooctanoic acid (PFOA); perfluorohexane sulfonate (PFHxS); poly fluorinated carboxylic acids, alkyl sulfonates; alkyl sulfonamido compounds; and fluorotelemeric compounds.

As used herein, a “pharmaceutical” refers to a compound manufactured, produced, extracted or otherwise obtained for use as a medicinal and/or therapeutic agent. Pharmaceuticals can be any molecule or molecules that are meant to be delivered into blood and/or lymphatic circulation, tissues, or organs, ultimately reaching a site in a subject's body where a positive impact on the subject's health, either locally or systemically, can be effected. These include, but are not limited to, muscle relaxants; digestive aids (e.g., reflux suppressants, laxatives, probiotics, prebiotics, and antidiarrheals); cardiovascular drugs (e.g., beta blockers, calcium channel blockers, diuretics, vasoconstrictors, vasodilators, cardiac glycosides, antiarrhythmics, nitrates); blood pressure/hypertension drugs (e.g., ACE inhibitors, alpha blockers, angiotensin receptor blockers); coagulation drugs (e.g., anticoagulants, heparin, antiplatelet drugs, fibrinolytics, anti-hemophilic factors and haemostatic drugs); statins (e.g., LDL cholesterol inhibitors and hypolipidaemic agents); endocrine aids (e.g., androgens, antiandrogens, estrogens, gonadotropin, corticosteroids, HGH, vasopressin); antidiabetics (e.g., sulfonylureas, biguanides, metformin, thiazolidinediones, insulin); thyroid hormones and antithyroid drugs; urogenital system drugs (e.g., antifungals, alkalinizing agents, quinolones, antibiotics, cholinergics, anticholinergics, hormonal contraceptives); central nervous system drugs (e.g., psychedelics, hypnotics, anesthetics, antipsychotics, eugeroics, antidepressants (including tricyclics, monoamine oxidase inhibitors, lithium salts, and SSRIs), antiemetics, anticonvulsants/antiepileptics, stimulants, amphetamines, dopamine agonists, antihistamines, cannabinoids, 5-HT antagonists); ocular medications (e.g., topical anesthetics, sympathomimetics, parasympatholytics, mydriatics, cycloplegics, mast cell inhibitors); antimicrobials (e.g., antibiotics, antibacterials, antifungals, antiparasitics, antiprotozoals, amoebicides); antivirals, antihistamines, anticholinergics, antiseptics, cerumenolytics, bronchodilators, antitussives, mucolytics, decongestants, antimalarials, antitoxins, antivenoms, vaccines, immunoglobulins, immunosuppressants, interferons, monoclonal antibodies, chemotherapeutic drugs and/or any other category of compounds that are capable of treating any health condition, disease or disorder, or of enhancing health in any way.

Non-limiting examples of pharmaceuticals according to the subject invention include atenolol, lisinopril, bisoprolol, carvedilol, labetalol, metoprolol, propranolol, sotalol, aspirin, naproxen, ibuprofen, acetaminophen, metformin, benzoyl peroxide, calamine (zinc oxide/ferric oxide), salicylic acid, dimethicone, hydrocortisone (cortisol), sunscreen (e.g., oxybenzone, avobenzone, octisalate, octocrylene, homosalate, or octinoxate), antacids/proton-pump inhibitors (e.g., bismuth subsalicylate, famotidine, lansoprazole, ranitidine hydrochloride, omepraole, calcium carbonate), insulin, antihistamines (e.g., brompheniramine, cetirizine, chlorpheniramine, clemastine, diphenhydramine, fexofenadine, loratadine), phenylephrine, pseudoephedrine, lotrimin, miconazole, clotrimazole, tinactin, ketoconazole, benzocaine; and antibiotics, including, for example, penicillins (such as penicillin G, penicillin V, ampicillin, amoxicillin, bacampicillin, carbenicillin, carbenicillin indanyl, ticarcillin, azlocillin, mezlocillin, methicillin, piperacillin, and the like), moxifloxacin, tetracyclines (such as chlortetracycline, oxytetracycline, methacycline, doxycycline, minocycline and the like), cephalosporins (such as cefadroxil, cephalexin, cephradine, cephalothin, cephapirin, cefazolin, cefaclor, cefamandole, cefonicid, cefoxitin, cefotetan, cefuroxime, cefuroxime axetil, cefinetazole, cefprozil, loracarbef, ceforanide, cefepime, cefoperazone, cefotaxime, ceftizoxime, ceftriaxone, ceftazidime, cefixime, cefpodoxime, ceftibuten, and the like), fluoroquinolones (e.g., levofloxacin), neomycin, polymyxin, quinolones (such as nalidixic acid, cinoxacin, ciprofloxacin and norfloxacin and the like), lincomycins (e.g., clindamycin), macrolides (e.g., erythromycin, azithromycin), sulfones (e.g., dapsone), sulfonamides (e.g., sulfanilamide, sulfadiazine, sulfamethoxazole, sulfisoxazole, sulfacetamide, bactrim), lipopeptides (e.g., daptomycin), polypeptides (e.g., bacitracin), glycopeptides (e.g., vancomycin), aminoglycosides (e.g., streptomycin, gentamicin, tobramycin, amikacin, netilmicin, kanamycin, and the like), nitoimidazoles (e.g., metronidazole) and/or carbapenems (e.g., thienamycin).

In certain embodiments, “pharmaceuticals” as used herein can also include health-promoting substances, such as vitamins, minerals and supplements, as well as personal care products and cosmetics.

As used herein, “remediation” means reducing the amount of a contaminant at a site by any amount, which can include complete purification or removal of the contaminant. In some embodiments, remediation is effected by breaking down, dissociating or degrading the contaminant into smaller, less harmful molecules or elements. In some embodiments, remediation is effected by converting the contaminant into a different, less harmful, substance. In some embodiments, such as in the context of a microbial contaminant, remediation is effected by controlling, killing and/or eradicating the microorganism, reducing the cell count, or inhibiting pathogenicity or further growth of the microorganism at the site.

As used herein, “reduction” means a negative alteration of at least: 1%, 5%, 10%, 20%, 25%, 30%, 35%, 40%, 45%, 50%, 55%, 60%, 65%, 70%, 75%, 80%, 85%, 90%, 95%, 98%, 99% or 100%.

### Photocatalyst Composition

In certain embodiments, the subject invention provides a stable, three-dimensional (3D) photocatalyst composition comprising a metal oxide semiconductor doped with, e.g., carbon derived from a carbon precursor material.

Metal oxide/sulfide semiconductors according to the subject invention include, for example, ZnO, TiO₂, NiO, Ni₂O₃, CuO, Cu₂O, Co₂O₃, W₂O₃, WO₃, V₂O₅, Fe₃O₄, FeO, FeO₂, Fe₂O₃, RuO₂, RuO₄, Re₂O₇, MoO₃, ZrO₂, Cr₂O₃, CrO, CrO₂, SnO₂, CeO₂, FeS₂, CdS, NiS, NiS₂, Ni₃S₂, Ni₃S₄, CoS, Bi₂S₃, ZnS, CuS, Cu₂S, PbS, MoS₂, and/or WS₂. In preferred embodiments, the metal oxide it TiO₂.

In some embodiments, a photocatalyst can be doped with at least one naturally occurring element to improve the activity of the photocatalyst. Such an element may be called a “dopant”. Dopants can be provided as precursors added generally during synthesis. Doped elements (dopants) can be elements that are incorporated into the crystal lattice of the Ti compound, for example as substituted within defined positions within the crystal lattice or otherwise interstitially included within the crystal. In some embodiments, the dopant can be selected from one of more elements including alkali metals such as lithium (Li), sodium (Na), potassium (K), and cesium (Cs); alkali earth metals such as magnesium (Mg), calcium (Ca), strontium (Sr), barium (Ba); noble metals such as gold (Au), platinum (Pt), rhodium (Rh), iridium (Ir), palladium (Pd), and ruthenium (Ru); transition metals such as iron (Fe), copper (Cu), zinc (Zn), vanadium (V), titanium (Ti) (for example for W-based compounds), tungsten (W) (for example for Ti-based compounds), manganese (Mn), Mo, zirconium (Zr), niobium (Nb), chromium (Cr), cobalt (Co), cerium (Ce) and nickel (Ni); lanthanide and actinide metals; halogens; Group III elements (from the Dmitri Mendeleev/Lothar Meyer style modern periodic table with elements arranged according to increasing atomic number) including B, Al, Ga, In and TI, Group IV elements including Ca, Si, Ge, Sn; Group V elements like N, P, As, Bi; and Group VI elements like S and Se. In some embodiments, the photocatalyst can be doped with at least one element selected from C, N, S, F, Sn, Zn, Mn, Al, Se, Nb, Ni, Zr, Ce and Fe. In some embodiments, the photocatalyst may be self-doped, e.g., Ti³⁺ in place of Ti⁴⁺ in a TiO₂ matrix.

In some embodiments, the photocatalyst can be formed using, as a dopant, dried lignin or another carbon precursor material comprising a renewable polymer of natural or synthetic origin. In one embodiment, the renewable polymer comprises chitosan, chitin, cellulose, guar, alginic acid, hemicellulose, dextran, xanthan, agar, lignin, starch, sucrose, dextrose, silk, wool, DNA, RNA, protein, glycogen, amylose, enzymes, polypeptides, pectin, keratin, collagen, hyaluronic acid or their derivatives and renewable synthetic polymers comprise of polylactic acid, polyglycolic acid, polycaprolactone, polyaspartic acid, polyphosphazenes, polyhydroxyalkanoate, polybutyrate, poly-3-hydroxybutyrate, poly(lactide-co-glycolide), poly(hydroxybutyrate-co-hydroxyvalerate), poly(butylene succinate), poly(butylene succinate adipate), poly(ethylene succinate), or a combination thereof.

In preferred embodiments, the carbon precursor material is lignin. The lignin or other carbon precursor material can be derived from any biomass material, preferably vegetal biomass, comprising cellulose, hemicellulose and/or lignocellulose, preferably comprising lignocellulose. Such biomass includes, but is not limited to, plant material such as forestry products, woody feedstock (softwoods and hardwoods), agricultural wastes and plant residues (such as corn stover, sorghum, sugarcane bagasse, grasses, rice straw, wheat straw, empty fruit bunch from oil palm and date palm, agave bagasse), perennial grasses (switchgrass, miscanthus, canary grass, erianthus, napier grass, giant reed, and alfalfa); plant-based municipal solid waste (MSW), aquatic products such as algae and seaweed, wastepaper, paper processing by-products, cotton, hemp, natural rubber products, and food processing by-products.

“Lignin,” as used herein, is the generic term for a group of aromatic cross-linked polymers present in the cell walls of support tissues of vascular plants and some algae. The main building blocks of lignin are the hydroxycinnamyl alcohols (or monolignols) coniferyl alcohol, sinapyl alcohol and p-coumaryl alcohol, which provide a 3D structural framework and hydroxyl groups suitable for chemical modification. As one of the most abundant biopolymers on earth, lignin presents a sustainable means to synthesize functional materials.

In certain embodiments, the lignin content of the photocatalyst is about 0.1% to about 75%, or about 0.5%, 1.0%, 5%, 10%, 15% or 50%, by weight. In preferred embodiments, the lignin content is about 0.5% to 15% by weight.

In some embodiments, the photocatalyst can function under light having a wavelength of about 10 nm to 850 nm, or about 100 nm to 820 nm. Advantageously, the photocatalyst composition can contribute to significant photodegradation activity under a broad range of electromagnetic radiation wavelengths (including visible light wavelengths) compared to known metal-based photocatalysts that operate only under ultraviolet (UV) wavelengths. That is, the photocatalyst has an advantage in that it can be used indoors and in natural sunlight.

As used herein, “ultraviolet light (UV light)” refers to light having a wavelength shorter than visible light and longer than X rays, that is, light which is in a wavelength range of approximately 10 to 390 nm and has energy higher than visible light.

As used herein, “visible light” refers to light having a wavelength shorter than infrared light and longer than UV light, that is, light that is in a wavelength range of approximately 390 to 820 nm, and accounts for the largest percentage among light (for example, ultraviolet light, visible light, infrared light, and the like) included in sunlight.

The photocatalyst may have a band gap that corresponds to light in the visible range, such as a band gap greater than about 1.5 eV, less than about 3.5 eV, about 1.5 eV to about 3.5 eV, about 1.7 eV to about 3.3 eV, or 1.77 eV to 2.33 eV. Some photocatalysts may have a band gap of about 1.2 eV to about 6.2 eV, about 1.2 eV to about 1.5 eV, or about 2.0 eV to about 6.2 electron eV.

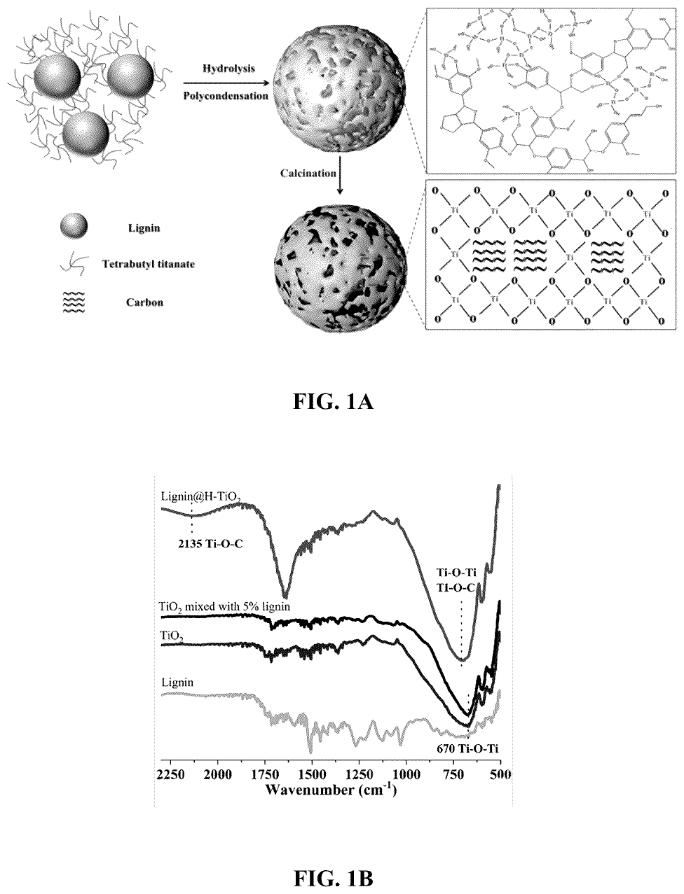

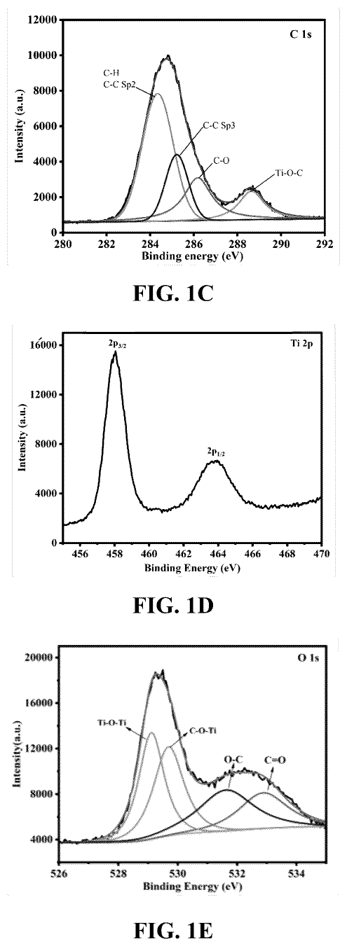

In a specific embodiment, the photocatalyst composition comprises a hollow, spherical or spheroid nanoparticle structure, which is formed by establishing stable Ti—O—C and Ti—O—Ti bonds through the crosslinking of lignin hydroxyl surface functional groups with hydrolyzed Ti(OH)₄. FIG. 1A. The average diameter of the nanoparticles can range from 5 to 1000 nm, from 100 to 900 nm, from 250 to 800 nm, from 400 to 700 nm, or from 500 to 600 nm.

In certain embodiments, the photocatalyst structure is formed by a lattice of bonds that allow for uniform and stable carbon modification of TiO₂, which can improve the resistance of the photocatalyst to photocorrosion and extend the lifespan of the photocatalyst over unmodified TiO₂. For example, in certain embodiments, the durable lattice structural design allows for stable photocatalyst performance under visible light irradiation that retains function for at least 5 cycles of usage, or preferably a least 10 cycles of usage. Accordingly, in some embodiments, the compositions of the subject invention can be reusable and/or recyclable.

Furthermore, in certain embodiments, the lattice nanoparticle shell structure of the photocatalyst can improve photo-utilization, including from visible light spectrum wavelengths, compared with unmodified TiO₂ photocatalysts. The photogenerated charge is stabilized due to efficient scattering of light throughout the hollow spherical cavity, which provides ample active sites relative to surface area. Furthermore, the 3D structure effectively separates the electrons and holes (i.e., the active species for photocatalysis), thereby inhibiting charge recombination and achieving highly efficient oxidative capacity in the visible light wavelength range.

In various embodiments, the photocatalyst may exhibit one or more of the characteristics of a Fourier Transform Infrared Spectroscopy (FTIR) spectra as depicted in FIG. 1B and/or one or more of the characteristics of an X-ray photoelectron (XPS) spectra as depicted in FIGS. 1C-1E.

The photocatalyst composition of the subject invention can be incorporated into a variety of formulations depending upon the desired usage. For example, in some embodiments, the photocatalyst composition is formulated as part of an aqueous and/or aerosol formulation; imbued into a substrate, such as a cleaning wipe, a membrane and/or an air or water filter; and/or affixed to a surface in the form of a film or coating.

Furthermore, the photocatalyst can comprise one or more additional components, for example, carriers (e.g., water), surfactants, hydrophilic and/or hydrophobic syndetics, sequestrants, builders, solvents (e.g., water, short-chain alcohols), organic and/or inorganic acids, beneficial microorganisms, botanical extracts, cross-linking agents, chelators, fatty acids, alcohols, reducing agents, oxidants, buffers, enzymes, dyes, colorants, preservatives, propellants, emulsifiers, demulsifiers, foaming agents, defoamers, bleaching agents, polymers, thickeners and/or viscosifiers.

Also provided herein are methods for producing a photocatalyst according to the subject invention, comprising mixing titanium (IV) n-butoxide with a dispersion of lignin nanoparticles; copolymerizing the titanium (IV) n-butoxide with the lignin nanoparticles via hydrolysis of the titanium (IV) n-butoxide; and calcining the resulting copolymer. FIG. 1A.

### Method of Remediating a Contaminant Using Photocatalyst

The subject invention further provides methods for remediating a contaminant from a site using a photocatalyst composition according to the subject invention.

In certain embodiments, the methods comprise contacting an effective amount of the photocatalyst composition with the contaminated site such that the photocatalyst is proximate to the contaminant and initiates and/or enhances the remediation of the contaminant upon irradiation with electromagnetic radiation. Photocatalysis results due to reactive species (able to perform reduction and oxidation) being formed on the surface of the photocatalyst from the electron-hole pairs generated in the bulk of the photocatalyst by the absorption of electromagnetic radiation. In preferred embodiments, the electromagnetic radiation is within the visible light spectrum.

In some embodiments, the methods further comprise measuring the amount of degradation achieved by the method after some period of time after the introduction of the photocatalyst in the presence of electromagnetic radiation.

An “effective amount” as used herein refers to an amount capable of remediating at least 50% of a contaminant at a site, or in a sample, within a time period of 12 hours, or preferably within 3 hours, or less, of contact with the photocatalyst in the presence of electromagnetic radiation. In certain embodiments, the amount of photocatalyst applied is about 1 to 150 mg, about 5 to 100 mg, about 10 to 75 mg, about 15 to 50, or about 20 to 30 mg per 1 ppb of contaminant.

Electromagnetic irradiation may be performed without particular limitation as long as the photocatalyst can be irradiated with a sufficient amount of light. In certain embodiments, light irradiation may be performed using light having a wavelength of 10 nm to 850 nm, and specifically light having a wavelength of 100 nm to 820 nm; 380 to 800 nm; 390 to 820 nm; 400 to 800 nm; 400 to 700 nm; 450 to 750 nm; 500 to 800 nm; 450 to 650 nm; 500 to 720 nm; 380 to 500 nm; or 380 to 450 nm.

The light may come from sunlight, lamps, lightbulbs, fluorescent/gas discharge lamps, ultraviolet and non-ultraviolet light emitting diodes, mercury vapor lamps, xenon lamps, halogen lamps, combination gas lamps, microwave sources or other known sources of visible or infrared illumination.

In certain embodiments, the method does not require the physical supplying of light in order provide the necessary energy for photodegradation. That is, in certain embodiments, the site of application is in the open environment where access to sunlight is readily available. In certain embodiments, the method can comprise supplying light to the photocatalyst after its application to the site, either in lieu of, or as a supplement to, natural sunlight.

In one embodiment, the photocatalyst is left at the site of application for a sufficient time to achieve remediation, for example, from 0.001 seconds to 30 days, from 0.01 seconds to 48 hours, from 0.1 seconds to 36 hours, from 1 second to 24 hoors, from 5 seconds to 12 hours, from 10 seconds to 8 hours, from 30 seconds to 6 hours, from 60 seconds to 3 hours. Preferably, the minimum exposure time required is less than 12 hours, more preferably less than 3 hours, in order to achieve at least 50% remediation.

In some embodiments, the photocatalyst is left at the site and can provide remediation for, minimally, at least 24 hours, preferably for up to 7 days, more preferably for up to 14 days, or most preferably for 30 days or longer, as measured from the time of application.

In one embodiment, the method further comprises the step of removing the photocatalyst and any dissociated materials that are left over from remediation of the contaminant. For surfaces, in particular, this can be achieved by, for example, rinsing with water, and/or rubbing or wiping with a cloth or sponge.

The photocatalyst composition can be used independently from, or in conjunction with, an absorbent and/or adsorbent material.

The subject methods can be used for remediation of various contaminants using various means of application. In one embodiment, the site can be in any physical state, including solid, liquid, gas, plasma or combination thereof for treatment. The target contaminant can be present at the site at, for example, less than 1.0 ppb to up to higher than 10,000 ppb. In certain instances, the site is completely, or almost completely, comprised of the contaminant, i.e., the contaminant is the site of application.

In certain embodiments, the photocatalyst can be used for remediating a contaminant from a fluid or liquid medium. The terms “fluid” or “liquid medium” are used herein to refer to a substance which is in the form of a liquid at ambient temperature or room temperature.

In one embodiment, the site is an aqueous treatment stream, such as liquid effluent, wastewater, industrial runoff and/or agricultural runoff. In one embodiment, the photocatalyst is used within a water processing system to treat/disinfect water, such as tap water, to remediate pollutants and microbial contaminants. In one embodiment, the photocatalyst can be used in a system to activate a stream of water, such as for clothes and dishwashing machines, to reduce reliance on chlorine bleach and reduce detergent consumption.

In certain embodiments, the contaminated site comprises solid and sludge waste sources, such as soil, animal manure or landfill waste.

The present invention can utilize both in situ and ex situ remediation methods of contaminated solids, soils, and fluids (e.g., ground and surface water), wherein in situ techniques are defined as those that are applied to, for example, soil and groundwater at the site with minimal disturbance. Ex situ techniques are those that are applied to, for example, soil and groundwater that have been removed from the site via, for example, excavation (e.g., soil) or pumping (e.g., water). In situ techniques are generally the most desirable options due to lower cost and fewer disturbances to the environment.

In some embodiments, bioreactors, tanks or columns, including slurry reactors or aqueous reactors, can be used for ex situ treatment of contaminated solids or fluids pumped from a contaminated site. In such embodiments, a fluid or slurry comprising a contaminant is contacted with the photocatalyst within the reactor to reduce the amount of contaminants found within the contaminated fluid or slurry. The remediated fluid or slurry can then be recovered from the tank or the column.

In certain embodiments, the columns or tanks can be of various capacities, including, for example, at least about 1 mL, about 10 mL, about 100 mL, about 1 L, about 2 L, about 5 L, about 10 L, about 100 L, about 1000 L, about 10000 L, about 100000 L, or about 1000000 L.

In certain embodiments, the method may further comprise mixing a radical carrier with the aqueous solution. The radical carrier is photodecomposed during water treatment to form hydroxyl radicals (·OH⁻). Then, the hydroxyl radicals thus formed may serve as a catalyst that decomposes the organic compound remaining in the aqueous solution. Radical carriers may be used without limitation as such a radical carrier as longs the radical carriers may stably form radicals by light irradiation in the aqueous solution. According to one example, hydrogen peroxide (H₂O₂) may be used.

In some embodiments, remediation in reactors involves the processing of contaminated solid material (e.g., soil, sediment, sludge) or fluids with the photocatalyst through an engineered containment system.

In certain embodiments, contaminated fluid from a particular site, such as, e.g., wastewater or combustion exhaust, is brought into contact with a substrate embedded or coated with a photocatalyst as described herein. In some embodiments, the substrate can be a porous PTFE (High-efficiency particulate absorption [HEPA]/ULPA Filter), other HEPA (e.g., those removing 99.97% of particles that have a size of 0.3 microns or larger) or HEPA like filters, non-woven or woven textile, a folding filter (Textile, paper, porous plastic as such as Porous PTFE), Glass/quartz wool, fiber (cellulose, glass quartz, plastics, resins), honeycomb structured cellulose, polymer, metal or ceramic, activated or porous carbon, zeolites (microporous aluminosilicates), or any existing filter materials.

Additional substrates can include, glass (e.g., windows, mirrors), walls (e.g., drywall), floors, joinery, stone (e.g., granite counter tops, flooring), masonry (e.g., brick walls), metals (e.g. stainless steel, metal alloys [handles, handrails, faucets]), natural fibers (e.g., cellulose, cotton), woods (e.g., furniture, fencing, shutters,), resin materials (plastics) such as polypropylenes (PP), polyethylenes (e.g., polyethylene [PE], polyethylene terephthalates (PET), polytetrafluoroethylenes (PTFE), polyvinylidene fluorides, polyimides and polyamide-imides, perfluoralkoxy polymer resins, fluorinated ethylene propylene (FEP), ethylene tetrafluoroethylene (ETFE) (e.g. plastic wrap for flowers, plastic handles, plastic keyboards elements), other polymeric surfaces, ceramics (e.g., porcelains [bathtubs, ceramic tiles, sinks, shower stalls, toilets]), other organic substrates (e.g., activated carbon), and the like.

In some embodiments, the substrate can be a gas or liquid permeable, or non-permeable, membrane made of, for example, a polyurethane.

The photocatalyst-embedded substrate can be utilized in, for example, bioreactors, gas masks, HVAC systems, vacuum cleaners, air filters, water filters, fuel tanks, dishwashers, humidifiers, dehumidifiers, and exhaust scrubbers.

In certain embodiments, the fixing of the photocatalyst onto or within a substrate can help control the amount of the photocatalyst the comes into contact with the contaminant, which may help prevent the rate of remediation from deteriorating due to a shielding effect caused by an excessive amount of the photocatalyst.

In some embodiments, pressure and/or stirring can be applied to force a contaminated fluid through the photocatalyst substrate. The pressure may be a positive pressure, which is provided by, for example, a positive displacement pump that is located upstream of and fluidly connected to a filter apparatus containing the photocatalyst substrate. Alternatively, the pressure may be a negative pressure, which is provided by, for example, a vacuum pump that is located downstream of and fluidly connected to the outlet of the filter apparatus. Each of the positive or negative pressure may be in a range of about 1 to about 10 bars, about 2 to about 8 bars, or about 4 bars.

In certain embodiments, the photocatalyst is applied to a site using a cleaning wipe, sponge (cellulose, synthetic, etc.), paper towel, napkin, cloth, towel, rag, mop head, squeegee, and/or other cleaning device that includes an absorbent and/or adsorbent material. The cleaning composition can be pre-loaded onto an absorbent and/or adsorbent material, post-absorbed and/or post adsorbed by a material during use, and/or be used separately from an absorbent and/or adsorbent material.

A cleaning wipe, upon which the photocatalyst composition can be loaded thereon, can be made of an absorbent/adsorbent material. Typically, the cleaning wipe has at least one layer of nonwoven material. Non-limiting examples of commercially available cleaning wipes that can be used include DuPont 8838, Dexter Z A, Dexter 10180, Dexter M10201, Dexter 8589, Ft. James 836, and Concert STD60LN. All these cleaning wipes include a blend of polyester and wood pulp. Dexter M10201 also includes rayon, a wood pulp derivative. The photocatalyst composition is loaded onto the cleaning wipe in any number of manufacturing methods. Typically, the cleaning wipe is soaked in the photocatalyst composition for a period of time until the desired amount of loading is achieved.

In certain embodiments, the photocatalyst is applied to a site as part of a liquid or semi-liquid formulation, for example, a dispersion, a colloid, an aqueous solution or an aerosol that can be poured, spread and/or sprayed onto the site.

In certain embodiments, the photocatalyst is applied to a site as part of a coating or film that can serve to prevent contamination of surfaces, including, but not limited to, mobile phones, light switches, doorknobs, walls, medical implants, medical devices, gym and sporting equipment, food packaging, cooking utensils, textiles, appliances, touch panels, touch screens, fluid storage containers, fuel tanks and other high-touch surfaces in public places, such as airplanes, motor vehicles, boats, public transport, bathrooms, kitchens, restaurants, playgrounds, stadiums and places of worship.

The coating or film may be formulated with, e.g., cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, methacrylic resins, low density polyethylene (LDPE), density polyethylene (HDPE), polypropylene (PP), polyvinyl chloride (PVC), polyethylene terephthalate (PET), polyethylene terephthalate glycol-modified (PETG), Nylon 6, ionomer, nitrile rubber modified acrylonitrile-methyl acrylate copolymer, and polysaccharides such as polyurethane. Additionally, the coating material may contain conventional carriers and additives such as plasticizers, pigments, colorants, glidants, stabilization agents, pore formers and surfactants.

### Additional Embodiments and Claimable Subject Matter

1. A photocatalyst composition comprising a metal oxide semiconductor modified with a carbon dopant derived from a carbon precursor material, wherein the photocatalyst composition comprises a hollow, nanoparticle structure having a spherical or spheroid shape, and wherein the photocatalyst composition is capable of facilitating remediation of a contaminant upon irradiation via light within the visible light range.

2. The photocatalyst composition of embodiment 1, wherein the metal oxide semiconductor is ZnO, TiO₂, NiO, Ni₂O₃, CuO, Cu₂O, Co₂O₃, W₂O₃, WO₃, V₂O₅, Fe₃O₄, FeO, FeO₂, Fe₂O₃, RuO₂, RuO₄, Re₂O₇, MoO₃, ZrO₂, Cr₂O₃, CrO, CrO₂, SnO₂, CeO₂, FeS₂, CdS, NiS, NiS₂, Ni₃S₂, Ni₃S₄, CoS, Bi₂S₃, ZnS, CuS, Cu₂S, PbS, MoS₂, and/or WS₂.

3. The photocatalyst composition of embodiment 2, wherein the metal oxide semiconductor is TiO₂.

4. The photocatalyst composition of embodiment 1, wherein the carbon precursor material is lignin, chitosan, chitin, cellulose, guar, alginic acid, hemicellulose, dextran, xanthan, agar, starch, sucrose, dextrose, silk, wool, DNA, RNA, protein, glycogen, amylose, enzymes, polypeptides, pectin, keratin, collagen, hyaluronic acid or their derivatives and renewable synthetic polymers comprise of polylactic acid, polyglycolic acid, polycaprolactone, polyaspartic acid, polyphosphazenes, polyhydroxyalkanoate, polybutyrate, poly-3-hydroxybutyrate, poly(lactide-co-glycolide), poly(hydroxybutyrate-co-hydroxyvalerate), poly(butylene succinate), poly(butylene succinate adipate), poly(ethylene succinate), or a combination thereof.

5. The photocatalyst composition of embodiment 4, wherein the carbon precursor material is lignin.

6. The photocatalyst composition of embodiment 5, comprising 5% to 15% lignin by weight.

7. The photocatalyst composition of embodiment 1, wherein the metal oxide semiconductor it TiO₂ and the carbon precursor material is lignin, and wherein the photocatalyst composition is formed by mixing titanium (IV) n-butoxide with a dispersion of lignin nanoparticles; subjecting the titanium (IV) n-butoxide to hydrolysis, thereby copolymerizing the titanium (IV) n-butoxide with the lignin nanoparticles; and calcining the resulting copolymer, thereby establishing a lattice of Ti—O—C and Ti—O—Ti bonds.

8. The photocatalyst composition of embodiment 1, having an average nanoparticle diameter of about 250 to 600 nm.

9. The photocatalyst composition of embodiment 1, having a band gap of 1.77 eV to 2.33 eV.

10. The photocatalyst composition of embodiment 1, further comprising a substrate fixed with the nanoparticle structure, wherein the substrate is a filter comprising porous PTFE, non-woven or woven textile, glass/quartz wool, cellulose, plastics, polymers, resins, metal, ceramic, activated or porous carbon, or zeolites.

11. The photocatalyst composition of embodiment 1, further comprising a substrate fixed with the nanoparticle structure, wherein the substrate is a membrane comprising polyurethane.

12. The photocatalyst composition of embodiment 1, further comprising a substrate fixed with the nanoparticle structure, wherein the substrate is a cleaning wipe.

13. The photocatalyst composition of embodiment 1, further comprising additives such as carriers, propellants, adhesives, plasticizers, pigments, colorants, glidants, stabilization agents, pore formers and surfactants.

14. The photocatalyst composition of embodiment 13, comprising an aqueous carrier.

15. A method for remediating a contaminant at a site, the method comprising contacting an effective amount of the photocatalyst composition according to any of embodiments 1 to 7 with the site and irradiating the photocatalyst with electromagnetic radiation.

16. The method of embodiment 15, wherein the electromagnetic radiation has a wavelength of 10 nm to 860 nm.

17. The method of embodiment 15, wherein the electromagnetic radiation has a wavelength of 380 nm to 820 nm.

18. The method of embodiment 15, wherein the photocatalyst composition is formulated as a dispersion, a colloid, an aqueous solution or an aerosol, and wherein the photocatalyst is contacted with the contaminated site via spreading, spraying or pouring.

19. The method of embodiment 15, wherein the photocatalyst composition is fixed to a substrate that contacts the contaminated site, wherein the substrate is a cleaning wipe, a matrix, a membrane or a filter.

20. The method of embodiment 15, wherein the contaminated site is a solid, liquid, gas, plasma or combination thereof.

21. The method of embodiment 15, wherein the contaminated site is a surface on a phone, light switch, doorknob, wall, medical implant, medical device, gym and sporting equipment, cooking utensil, textile, appliance, touch panel, touch screen, fluid storage container, fuel tank, food packaging, or food.

22. The method of embodiment 15, wherein the contaminated site is a solid surface and wherein the photocatalyst is affixed to the surface in the form of a film or coating.

23. The method of embodiment 15, wherein the contaminated site is an aqueous treatment stream including liquid effluent, wastewater, industrial runoff and/or agricultural runoff.

24 The method of embodiment 15, wherein the contaminated site is a water processing system used to remediate tap water.

25. The method of embodiment 15, wherein the contaminated site is a solid waste source comprising soil, sewage, animal manure and/or landfill waste.

26. The method of embodiment 15, performed ex situ in a bioreactor, a tank or a column.

27. The method of embodiment 15, wherein the contaminant is a pathogenic or undesirable microorganism, a pharmaceutical, or a persistent organic pollutant.

28. The method of embodiment 27, wherein the pharmaceutical is atenolol, lisinopril, bisoprolol, carvedilol, labetalol, metoprolol, propranolol or sotalol.

29. The method of embodiment 27, wherein the pathogenic microorganism is a Pseudomonas sp. bacterium.

30. The method of embodiment 27, wherein the persistent organic pollutant is one or more of: aldrin, chlordane, dieldrin, endrin, heptachlor, HCB, mirex, toxaphene, PCBs, DDT, dioxins, polychlorinated dibenzofurans and perfluoroalkyl and polyfluoroalkyl substances (PFAS).

31. The method of embodiment 27, wherein the persistent organic pollutant is a PFAS selected from one or more of the following: perfluorooctane sulfonate (PFOS); perfluorooctanoic acid (PFOA); perfluorohexane sulfonate (PFHxS); perfluoroheptanoic acid (PFHpA); perfluorohexanoic acid (PFHxA); poly fluorinated carboxylic acids, alkyl sulfonates; alkyl sulfonamido compounds; and fluorotelemeric compounds.

32. The method of embodiment 15, wherein an amount of the contaminant at the site is reduced by at least 50% in a time period less than 12 hours from contact with the photocatalyst composition.

### Materials and Methods

Method of Preparing Photocatalyst “C_{lignin}@H—TiO₂”

Colloidal lignin particles were first initiated by dissolving 2 g of kraft lignin (dry basis) in 200 mL of acetone/water 3:1 (v/v) mixture and stirred for 3 h, followed by filtration using a glass microfiber filter (pore size 0.7 μm) to remove the undissolved lignin. The obtained solution was rapidly poured into 400 mL of MilliQ-water under vigorous stirring for 3 h. Acetone was further removed by reduced pressure distillation at 40° C. to obtain the lignin nano-particles dispersions. The dispersions of lignin nano-particles were freeze-vacuum dried and then stored in a desiccator for further characterization and use.

To prepare Lignin@H—TiO₂, 340 mg titanium (IV) n-butoxide was added into 50 mL lignin nano-particles ethanol dispersion with magnetic stirring for 3 h until a clear brown uniform solution was obtained. Subsequently, 10 mL MilliQ-water was added dropwise into the above solution, which gradually turned into an opaque brown-yellow dispersion due to the hydrolysis reaction of titanium (IV) n-butoxide. The opaque brown-yellow solution was kept reacting for 12 h, ensuring the complete hydrolysis of the titanium (IV) n-butoxide and its successful copolymerization with lignin. After reaction, the dispersion was centrifuged with ethanol for 2 times and MilliQ-water for 5 times to remove any unreacted lignin and residual ethanol. The resulting precipitate was subsequently dried at 60° C. for 3 h, yielding brown powder identified as Lignin@H—TiO₂.

To obtain Lignin@H—TiO₂ with different grafted lignin ratio, 0.4 mg, 0.8 mg, 4 mg, 8 mg, 12 mg and 50 mg lignin nano-particles were used to prepare Lignin@H—TiO₂ samples with lignin contents of 0.5%, 1.0%, 5%, 10%, 15% and 50%, respectively.

Lignin@H—TiO₂ were calcined into C_{lignin}@H—TiO₂ in a split tube furnace with a vacuum system. The thermostabilization process was carried out with heating from room temperature to 200° C. at a heating rate of 5° C./min and then holding at 200° C. for 20 min. Afterwards, the thermostabilized Lignin@H—TiO₂ were carbonized under nitrogen atmosphere (240 cm³/min). The temperature was increased from 200° C. to calcination temperature with a heating rate of 5° C./min. The holding time at calcination temperature was 1.5 h.

After the calcination process, a black powder was obtained as C_{lignin}@H—TiO₂. For the investigation of the impact of calcination temperature, various calcination temperatures within the range of 550° C. to 800° C. were employed.

Calcinated TiO₂ (H—TiO₂) is prepared with the same synthesis process, without adding lignin. TiO₂ without calcination was synthesized by dropping 340 mg titanium (IV) n-butoxide into 50 mL ethanol and dispersion with magnetic stirring for 3 h. After baking at 80° C. for 24h, a white powder was obtained.

### Characterization

To examine the presence of chemical functional groups, fourier transform infrared spectroscopy (FTIR) were generated using Thermo Nicolet 380 FTIR spectrometer in the wavelength range from 400 to 4000 cm 1. Chemical element and chemical bonding were examined based on the X-Ray photoelectron spectroscopy (XPS) spectra developed using Omicron XPS/UPS system with Argus detector and the Omicron's DAR 400 dual Mg/Al X-ray source (Mg, power of 300 W, SCR_022202). Scanning electron microscopy (SEM) images were recorded on an ultra-high resolution field emission scanning electron microscope (JEOL JSM-7500F, SCR_022202) equipped with a high brightness conical FE gun and a low aberration conical objective lens at an accelerating voltage of 5 kV. Transmission Electron Microscopy (TEM) and Energy-Dispersive X-ray Spectroscopy (EDS) were used to analyze the samples by using the Titan Themis 300 microscopes (SCR_022202).

The samples were prepared by dispersing the dry powders of the nanoparticles in ethanol through ultrasonication for one minute. The dispersion of each sample was deposited dropwise on the 400 mesh copper grids with an ultrathin carbon film of lacey carbon and dried in an open atmosphere.

Particle sizes were investigated with a Malvern DLS zeta potential analyzer at ambient temperature using ethanol as the solvent. All data were averaged over 6 cycles with 10 scans for each cycle. UV-vis diffuse reflectance spectra (UV-vis ERS) were measured on a Hitachi U4100 UV-vis-NIR spectrophotometer (Japan) with a Praying Mantis accessory. Thermogravimetric analysis (TGA) test was performed on a TGA 5500 thermogravimetric analyzer. About 5.0 mg of sample was heated from room temperature to 800° C. at a heating rate of 10° C./min under the N₂ atmosphere. All data were processed with software Origin 2022. EPR were texted with Bruker Elexsys E500 console with a standard resonator and CoolEdge cryo system.

### Band Energy Calculation

The band gap energy (Eg) of C_{lignin}@H—TiO₂ was calculated from the UV-vis spectrum using the Tauc Plot method, by analyzing the linear relationship between (αhν)^{1/2} and photon energy hν.¹ In these plots, the Eg values were obtained from the intercept of the extrapolation of the linear branch with the abscissa.

Preparation of C_{lignin}@H—TiO₂ Membrane

0.5 g binary mixture of C_{lignin}@H—TiO₂ and water-borne polyurethane was carefully transferred onto a glass slide. The coated glass slide was then cured under 80° C. for 10 minutes. After the mixture was fully cured, the coated glass slide was put into methanol and finally the C_{lignin}@H—TiO₂ membrane was obtained by peeling it off from the glass slide.

### Strain Construction and Culture

The plasmid roGFP2-Orp1 that expresses fused green fluorescent protein (roGFP2) and hydrogen peroxide (H₂O₂)— sensitive proteins (glutaredoxin 1 or Orp1) was used to investigate intracellular redox states of microorganisms.²¹ To construct the engineered strain, roGFP2-Orp1 plasmid was transformed into Pseudomonas putida A514 competent cells via electroporation using Bio-Rad Gene Pulser Electroporation system with parameters of 2.0 kV, 200 Omega, and 25 μF. The successful transformants were selected on a LB agar plate with 100 mg/L ampicillin using fluorescence as marker. Then Pseudomonas putida A514 with oxygen reduction potential (ORP) capabilities was cultured in Luria-Bertani (LB) medium overnight at 30° C. under agitation at 200 rpm. Following the culture period, the bacterial cells were harvested through centrifugation and subsequently re-suspended in phosphate-buffered saline (PBS) to achieve an optical density at 600 nm (OD600) of 0.2.

### Contaminant Photodegradation

10 mg C_{lignin}@H—TiO₂ was added in 5 mL porcelain crucibles with 2 mL PFAS solution or atenolol solution (2.5 ppm concentration). After 5 min adsorption equilibrium, the porcelain crucibles were placed under a solar simulator equipped with a xenon lamp (CME-SL500, Microenerg Beijing Technology Co., Ltd) with an intensity of 1800 W/m² and a working distance of 15 mm to test the photodegradation performance. After a certain time light treatment, a 40 μL sample solution was collected from the porcelain crucibles. and then 460 μL HPLC grade water was added into the above collected PFAS or atenolol solution to dilute the sample concentration to lower than 200 ppb for fitting the calibration curve. The HPLC-MS was used to detect the PFOA and PFOS concentration. Control samples were prepared in the same manner but without light treatment or without C_{lignin}@H—TiO₂ modification.

### Quantitative PFAS and Atenolol Analysis by High-Pressure Liquid Chromatography-Mass Spectrometry (LCMS)

Filtered PFAS or atenolol sample solutions (10 μL) were loaded into a 3.0 mm×50 mm (1.7 μm) Acquity UPLC BEH C18 column (Waters, MA, USA) to separate the compounds. An ammonium acetate aqueous solution (20 mM, solvent A) and 100% methanol (solvent B) were used as mobile phases, with a flow rate of 300 μL/min. The LC gradient started with 95% solvent A and 5% solvent B, and this ratio was kept until 1.00 min, then increased solvent B to 100% until 6.00 min, and kept the ratio until 7.00 min. Subsequently, the ratio was changed to 5% solvent B and 95% solvent A until 15.00 min. The mass spectrometer TSQ Quantiva (Thermo Fisher Scientific, San Jose, CA) was operated with a high temperature ESI source in negative mode. For PFAS, the ion source related parameters were: spray voltage: static; negative ion: 3219 V; sheath gas: 38.3 Arb; aux gas: 1.2 Arb; sweep gas: 2.8 Arb; Ion transfer tube temp: 325° C.; vaporizer temp: 50° C.; CID gas: 1.5 m Torr. For atenolol, the ion source related parameters were: spray voltage: static; positive ion: 4281 V; sheath gas: 38.3 Arb; aux gas: 1.2 Arb; sweep gas: 2.8 Arb; Ion transfer tube temp: 325° C.; vaporizer temp: 50° C.; CID gas: 1.5 mTorr. The calibration solutions were diluted with water to the corresponding concentration. Both calibration solutions and samples included internal standards with a spiked concentration of 5 μg/L.

### Plasmid Photodegradation

0.5 mg of C_{lignin}@H—TiO₂ was mixed with 20 μL of pLM231 at a concentration of 50 ng/uL (equivalent to 1 μg of DNA). And then the mixture was exposed to solar light for 5 to 30 min. The control groups include samples of anatase TiO₂ (purchased from Sigma)+pLM231, TiO₂ without calcination+pLM231, active carbon+pLM231, and C_{lignin}@H—TiO₂+pLM231 (subjected to dark treatment). The quantities of materials and the concentration of plasmid remain consistent across all groups. For the experimental group, 1 μL of the sample was taken out every 5 min during the light treatment period. Following the treatment, gel electrophoresis was performed to assess the concentration of the DNA plasmid.

### Antibacterial Performance for Coating Conditions

A binary mixture containing C_{lignin}@H—TiO₂ (100 mg) and waterborne polyurethane (5 g) was introduced into a 15 mL beaker. The mixture was subjected to magnetic stirring at 500 rpm for 5 min. Subsequently, this mixture was carefully transferred into a printing frame positioned atop a phone case. The printed pattern was then squeegeed by a scraper for three times, and cured under 80° C. for 20 min. Pseudomonas putida A514 ORP was inoculated in LB media overnight at 30° C. with agitation at 200 rpm. The bacterial culture was collected via centrifugation, and subsequently resuspended in PBS buffer to achieve an OD 600 of 0.2. The printed phone case was placed under a solar simulator and 20 μL of the above Pseudomonas putida A514 ORP solution was dropped on the printed and un-printed parts, respectively and then the setup was treated under light for 2 min. After light stimulation, the bacterial solutions from the printed and un-printed part were collected and diluted to a concentration of 1×10⁻⁵. The experiments were repeated for three times. Subsequently, 200 μL of the cultured solutions were evenly spread onto Luria-Bertani agar plates supplemented with ampicillin (LB^{Amp}). The agar plates were incubated at 30° C. overnight to facilitate colony enumeration.

### Antibacterial Performance in Solution

Pseudomonas putida A514 was inoculated in LB media with OD 600 of 0.2, and then the Pseudomonas putida solution was diluted into 1×10⁻⁵. Each 2 mL of the above solution was put into sealed clear glass bottles with 30 mg, 45 mg and 60 mg C_{lignin}@H—TiO₂, respectively. Then the 3 samples were put under a plant growth light (XS2000 LED Grow Light, 200 W, Viparspectra) for 1 h. Afterwards, 200 μL of the light treated Pseudomonas putida solutions were taken out and evenly distributed onto Luria-Bertani agar plates supplemented with ampicillin (LB^{Amp}). The agar plates were then incubated at 30° C. overnight, allowing for the growth of bacterial colonies. As a reference, control samples of Pseudomonas putida solutions with and without light were also tested.

### Steady-State Fluorescence

Steady-state photoluminescence emission and excitation spectra were recorded using RF-6001 fluorometer from Shimadzu. The films deposited on quartz slides were positioned at 45 degree in respect to excitation/emission slits. The widths of excitation and detection slits were set to 5 nm. To minimize effect of reflected and scattered light, 375 nm long-pass filter was placed at the detection entrance. All experiments were performed at room temperature in ambient air atmosphere.

### Transient Absorption

Time-resolved pump-probe absorption (transient absorption) experiments were carried out using Helios-EOS, a femtosecond/nanosecond tandem transient absorption spectrometer (Ultrafast Systems, USA) coupled to a femtosecond laser system (Spectra-Physics, USA), consisting Solstice, a one box ultrafast amplifier (Spitfire Pro XP—a Ti: sapphire regenerative amplifier with a pulse stretcher and compressor, Mai-Tai, a femtosecond oscillator and Empower, a diode-pumped solid state pulsed green laser). The laser system generates pulses at 800 nm with energy of ˜3.5 mJ, 1 kHz repetition rate and ˜90 fs duration. The output beam was split to 90 and 10%. Pump beam generated from 90% split was done by Topas Prime, an optical parametric amplifier (Light Conversion Ltd, Lithuania). The remaining 10% is used to produce probe pulses in the Helios spectrometer. For measurements in the EOS spectrometer (μs-ms delay time-range), probe pulses (˜1 ns duration) are generated by built-in PCF based supercontinuum pulsed light source. All samples were excited at 350 nm with energy of 1 μJ with excitation beam focused to 1 mm circular spot (2.3×10¹⁴ photons/cm², ˜0.1 J/cm⁻² fluence). To provide an isotropic excitation of the sample and avoid pump-probe polarization effects the pump beam was depolarized with achromatic depolarizer (DPU-25, Thorlabs). All experiments were performed at room temperature in ambient air atmosphere.

### Data Processing and Analysis of Transient Absorption (TA) Datasets

TA datasets were globally fitted with a kinetic model assuming (if more than one spectro-kinetic component was present) irreversible sequential decay of photoexcited species in slower steps, giving the so-called evolution associated difference spectra (EADS). According to this model, the TA signal at any time delay t, and wavelength λ, ΔA(t, λ), is reconstructed from the superposition of the n^{th} Cᵢ(t) and EADSᵢ(λ) products according to the formula:

$$Δ A ( t , λ ) = {\sum }_{i = 1}^{n} {C}_{i} ( t ) {EADS}_{i} ( λ ) \qquad ( 1 )$$

where Cᵢ(t) is the time-dependent concentration of the i^{th} EADS defined as:

$$\frac{d {C}_{i} ( t )}{dt} = {k}_{i - 1} {C}_{i - 1} ( t ) - {k}_{i} {C}_{i} ( t ) , i \neq 1 , {k}_{i - 1} > {k}_{i} \qquad ( 2 )$$

kᵢ is the rate constant of EADSᵢ, and Cᵢ₌₁(t) is populated by the excitation pulse represented by the instrument response function, IRF:

$$\frac{d {C}_{1} ( t )}{dt} = IRF - {k}_{1} {C}_{1} ( t ) \qquad ( 3 )$$

For fitting purposes, the IRF was simulated by a Gaussian with a full width at half maximum (FWHM) of ˜800 ns. The kinetic analysis was done using CarpetView software (Light Conversion Ltd., Lithuania).

### Photocurrent and Electrochemical Impedance Spectroscopy

Fluorine tin oxide (FTO) glass was employed as the transparent conductive substrate for the working electrode, which was cleaned by sonication in acetone, isopropanol, and water sequentially, followed by being blown with nitrogen gas to dry before use. Samples were suspended in ethanol by sonicating for 1h to produce a solution, which were then spray-coated onto an FTO glass and dried before test. The Photoelectric analysis was performed on a Bio-Logic SP 150e potentialstat utilizing a three-electrode photoelectrochemical cell with the FTO glass, a Pt wire, and an Ag/AgCl electrode as the working, counter and reference electrodes, respectively, in an aqueous electrolyte of 0.5 M Na₂SO₄. The area of the sample on the FTO exposed to light was 1 cm². The EIS measurements were performed over a range from 0.01 to 100 kHz at 0 V versus OCP, and the amplitude of the applied potential in each case was 10 mV.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

### Example 1—C_{lignin}@H—TiO₂ Preparation and Characterization

The lignin constructed C_{lignin}@H—TiO₂ photocatalyst was prepared via an environmentally friendly sol-gel method. FIG. 1A. Specifically, lignin was dispersed freely in ethanol due to the —OH functional groups outside the particle surface. Ti (OH) 4 was formed when tetrabutyl titanate slowly hydrolyzed with ethanol. Consequently, the TiO₂ growth was positively influenced by the reticular molecular structure of lignin as the hydrolysis reaction happened between the —OH groups of lignin and Ti (OH)+, forming the Ti—O—C and Ti—O—Ti crosslinking structures.¹²

The shell chemical structures of the 3D hollow particles were verified by Fourier-transform infrared spectroscopy (FTIR) and X-ray photoelectron spectroscopy (XPS) as shown in FIGS. 1B-1E. Lignin was uniformly distributed among the Ti—O—C and Ti—O—Ti crosslinking structures after the polymerization reaction, which ensured a durable carbon-doped TiO₂ structure. The peaks (FIG. 1D) at the binding energies of 458 and 464 eV can be attributed to Ti 2p_{3/2} and Ti 2p_{1/2}, respectively, suggesting that the valence state of Ti is +4.¹³

The O 1s XPS spectrum is shown in FIG. 1E and can be divided into four peaks. The peak at 531.6 eV and 532.9 eV are ascribed to C—O and C═O bonds, respectively. The peaks at 529.15 eV are commensurate with oxygen anions in the lattice (Ti—O—Ti), and the peak at 529.7 eV is attributed to C—O—Ti bonds.^{14.15} The dispersion structure obtained through grafting thus increased the 3D structure stability compared to other carbon modification structures.¹⁶ After calcination, the crosslinked lignin transformed into the carbon framework for the photocatalyst.

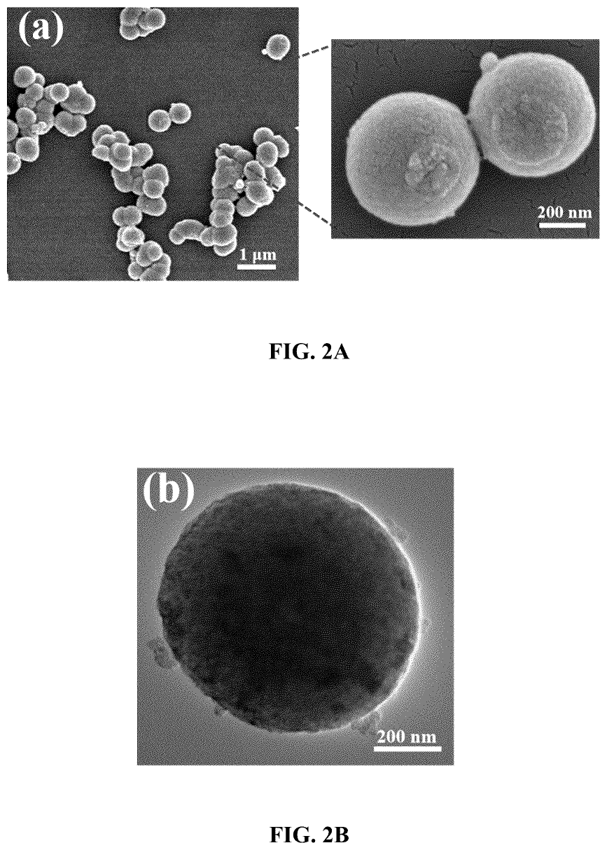

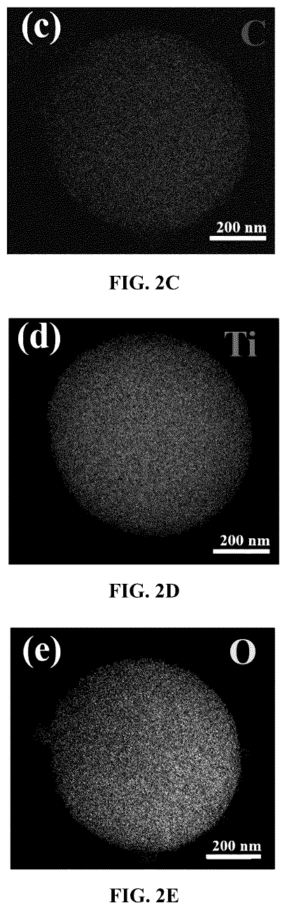

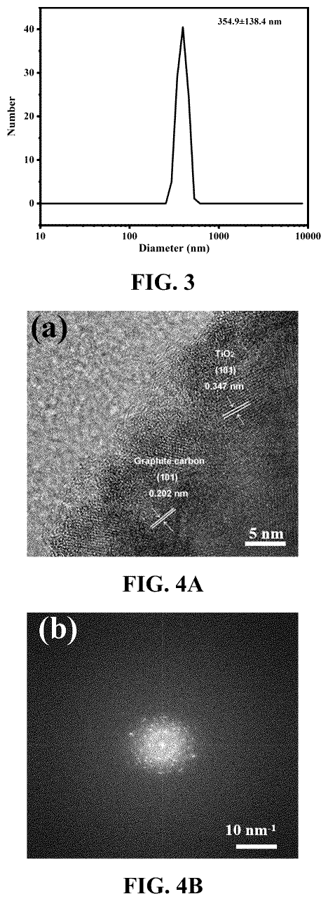

The morphology and microstructure of C_{lignin}@H—TiO₂ with 5% lignin were revealed through scanning electron microscopy (SEM) and transmission electron microscopy (TEM) analysis. FIG. 2A shows the field emission scanning electron microscopy (SEM) images of the prepared C_{lignin}@H—TiO₂. The particle diameters ranged from 250 to 600 nm with a mean value of about 350 nm from the SEM measurements and DLS test (FIG. 2A and FIG. 3). The TEM images of C_{lignin}@H—TiO₂ were shown in FIG. 2B, suggesting that the spherical structure was rather uniform. The transparent appearance could be due to the hollow structure.

As for a photocatalyst, the relative uniform size and rough structure could be beneficial for light harvesting. The lattice fringes and the crystal pattern were clearly visible (FIGS. 4A-4B), and the inter-planar distance was about 0.347 nm on average, which could be attributed to the (101) plane of anatase TiO₂.¹⁷ In addition to the covalent Ti—O—C, the (101) surface of TiO₂ also directly attached with the (101) surface of graphite carbon via weak van der Waals force to form a stable contact interface.¹⁸

Likewise, on the C_{lignin}@H—TiO₂ interface, the lattice fringes for the TiO₂ surface became disordered, possibly due to the presence of a certain orbital hybridization between the π and O 2p orbitals,¹⁹ indicating that the TiO₂ and the carbon were in tight contact, which was conducive to charge carriers between the two substances. Moreover, the X-ray (EDX) mapping image of the corresponding spherical particle of C_{lignin}@H—TiO₂ showed that Ti, O and C were homogeneously distributed in the shell (FIGS. 2C-2E), further suggesting a close hetero-interfacial contact between the carbonized lignin and TiO₂, and a uniform binary carbon hybrid nanoarchitecture due to the polymerization reaction.

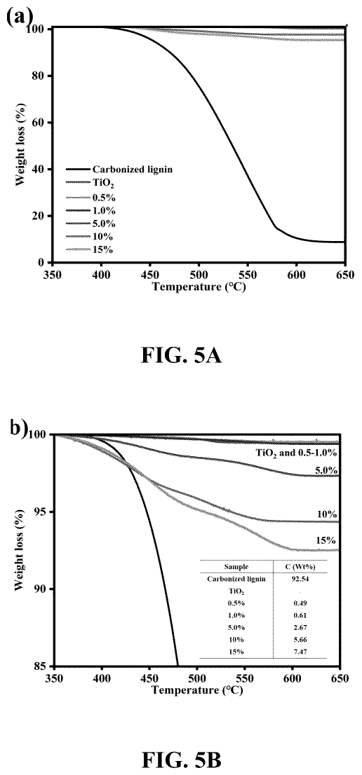

TG analysis was conducted to investigate the C_{lignin} content of C_{lignin}@H—TiO₂ synthesized with varying amounts of lignin. FIGS. 5A-5B. The initial weight loss observed from approximately 400 to 580° C. was due to the oxidation of carbon materials. A slight weight loss was observed for anatase TiO₂ from 520 to 550° C., which could be attributed to the decomposition of titanium-bonded groups such as —OH.¹¹ As the weight loss due to the decomposition of titanium-bonded groups was negligible,^{11, 20} the C_{lignin} content of C_{lignin}@H—TiO₂ synthesis with 5% lignin was estimated to be 2.67%.

### Example 2—Degradation Performance of C_{lignin}@H—TiO₂

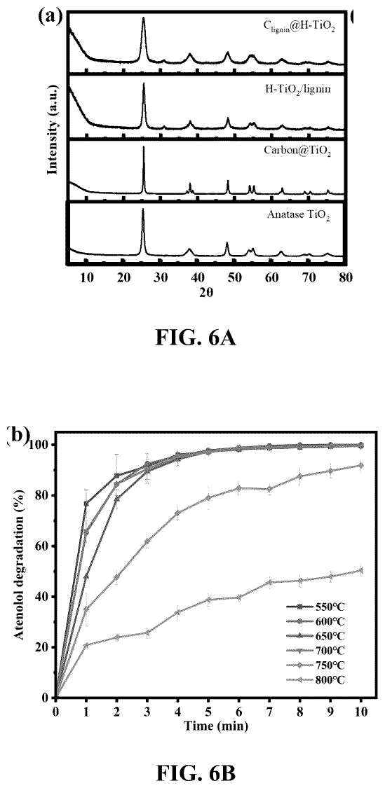

Using solar light irradiation, the photocatalytic performance of C_{lignin}@H—TiO₂ was evaluated by monitoring the photodegradation of atenolol, a model pharmaceutical compound. To show the effectiveness of the structural functional design, three other material designs were compared, which included H—TiO₂ mixed with carbonated lignin (H—TiO₂/C_{lignin}), Carbon@TiO₂ synthesized by adding boric acids, according to a previous report,²¹ and anatase TiO₂. All those materials showed anatase phase crystalline structure upon calcination at 600° C. for 90 minutes. FIG. 6A.

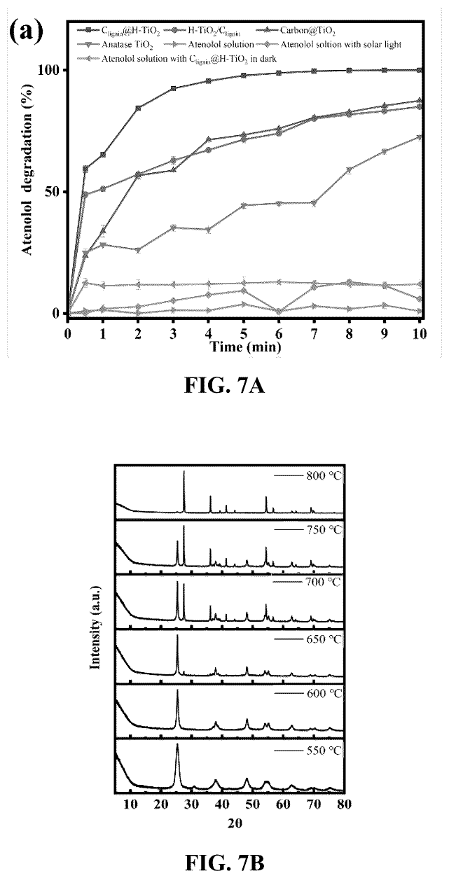

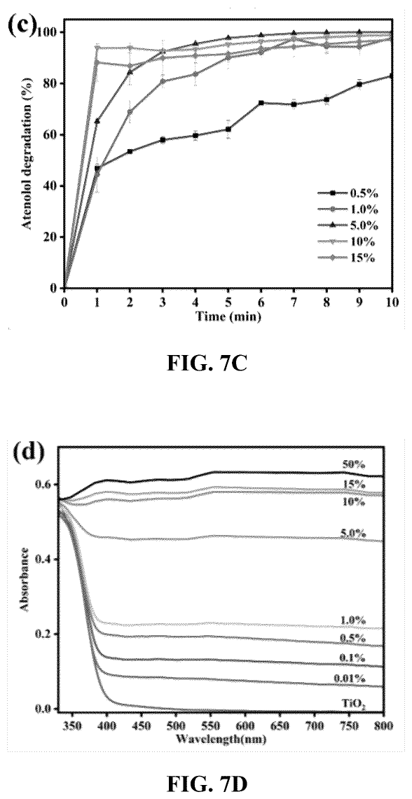

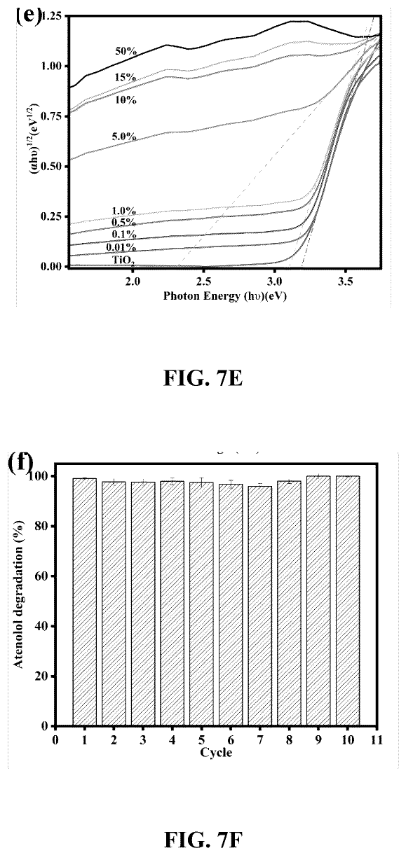

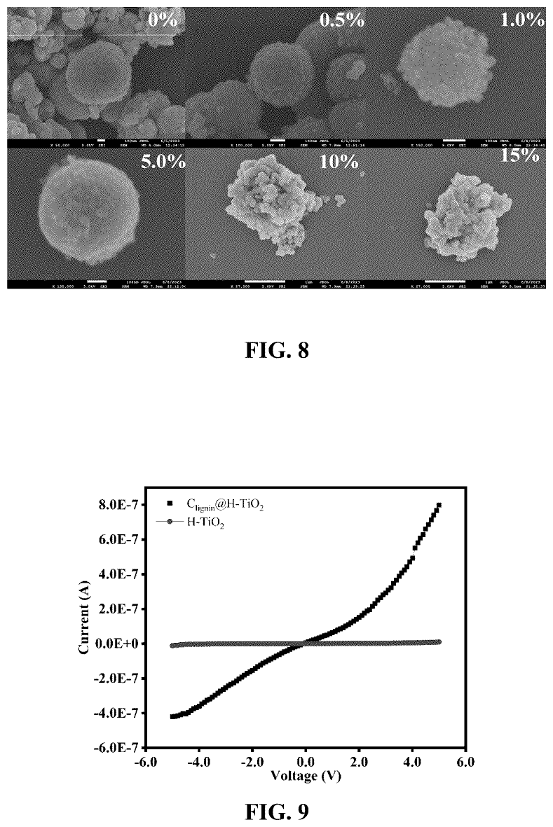

Before the degradation performance testing, the atenolol solution and atenolol solution mixed with the C_{lignin}@H—TiO₂ were kept in the dark. The atenolol solution with solar light irradiation was compared as the control. FIG. 7A.

In the ten minute testing period, the atenolol control was stable without degradation under solar light irradiation. Compared with other materials, C_{lignin}@H—TiO₂ showed the highest and fastest degradation rate, demonstrating the effective structural functional design with homozygous carbon-doped TiO₂ hollow particle structure. For example, the carbon-based Carbon@TiO₂ only reached 75% removal efficiency compared to that of C_{lignin}@H—TiO₂, which almost reached 100% with five minutes solar irradiation. The lignin and TiO₂ crosslinking structure of C_{lignin}@H—TiO₂ assembled C_{lignin}-doped mesoporous TiO₂ while transforming lignin to carbon during calcination treatment.²² Consequently, the solar lights likely transmitted through the holes and form multiple reflectances inside the hollow structure, enhancing the photocatalytic activity.

TABLE 1
Atenolol degradation performance of TiO₂ based photocatalytic materials

| | | | | | Degradation | |
|---|---|---|---|---|---|---|
| Catalytic | Light | Degradation | | Degradation | environmental | |
| material | source | time | | rate | solvent | Reference |
| TiO₂ (degussa P25) | 9 W UVA lamp | 30 | min | 67% | Ultrapure | 2 |
| | | | | | water | |
| Carbon dot/TiO₂ | 1000 W Xenon | 180 | min | 89% | Water | 3 |
| composite | lamp | | | | | |
| Alginate supported | UVA tube having | 60 | min | 58% | Water, | 4 |
| TiO₂ nanoparticles | 365 nm | | | | pH 10.5 | |
| | wavelengths | | | | | |
| Slurry suspension | UV radiometer of | 33.4 | min | 100% | Water | 5 |
| of TiO₂ (degussa P25) | 30 W m⁻² | | | | | |
| TiO₂ (degussa P25) | 1000 W Xe—OP | 120 | min | 63% | Ultrapure | 6 |
| | lamp | | | | water | |
| Graphene oxide- | 1000 W Xe arc | 60 | min | 72% | Deionized | 7 |
| TiO₂ composite | lamp and an AM | | | | water | |
| | 1.5G filter | | | | | |
| Heterojunction S-Tyr- | 300 W Xenon lamp | 120 | min | 95.46% | Deionized | 8 |
| NDI-Tyr/TiO₂ | equipped with a | | | | water | |
| | 420 nm filter | | | | | |
| TiO₂ (degussa P25) | High pressure | 60 | min | 100% | Milli-Q | 9 |
| | Mercury lamp | | | | water | |
| | (Philips, HPK, | | | | | |
| | 125 W) | | | | | |
| Aeroxide ® TiO₂ P25 | UVA radiation, 28 | 75 | min | 100% | Distilled | 10 |
| | W m² | | | | water | |
| C_{lignin}@H—TiO₂ | Solar simulator | 5 | min | 100% | Deionized | Present |
| | with 1800 W/m² | | | | water | disclosure |
| | Xenon lamp | | | | | |

As shown in Table 1, previous reports have applied various light sources using the TiO₂ based photocatalysts. The present design of C_{lignin}@H—TiO₂ showed the fastest efficiency even with solar light. Furthermore, C_{lignin}@H—TiO₂ removed all atenolol from the solution phase within the first five minutes, in which the rapid kinetics could be attributed to both the enhanced photocatalysis due to the hollow structure and increased surface area for adsorption.

As such, the BET surface area was measured for the synthesized materials. The surface areas were measured to be 30.2, 41.9, 51.8, 50.1, 132.4 and 147.5 m²/g for the samples pure TiO₂, C_{lignin}@H—TiO₂ with 0.5 wt %, 1.0 wt %, 5.0 wt %, 10 wt %, 15 wt % of lignin, respectively. The increased surface area could contribute to improved chemical adsorption and photocatalytic degradation.

Pore volumes were calculated to be 91.9, 102.6, 95.0, 110.8, 84.4 and 79.4 cm³/g·Å for the samples pure TiO₂, C_{lignin}@H—TiO₂ with 0.5 wt %, 1.0 wt %, 5.0 wt %, 10 wt %, 15 wt % of lignin, respectively. Nevertheless, H—TiO₂/C_{lignin} reached about 75% degradation for five minutes of solar irradiation and 80% degradation for ten minutes of solar irradiation, with much better performance than the individual anatase TiO₂.

### Example 3—Effects of Calcination on Photocatalyst Performance

To investigate the effects of calcination on the as-designed sample performance, XRD measurements were conducted to analyze their crystalline structures. FIG. 7B illustrates the formation of crystalline structures upon calcination for 90 minutes. When synthesized with 550° C. calcination, the sample showed much more anatase phase than the brookite phase. With the calcination temperature increase, the crystalline structure gradually changed to the anatase phase and finally reached the rutile phase. The rutile TiO₂ exhibited its most pronounced orientation preference along the (110) plane, which appeared at 2θ=27.44°, as well as the second and third strongest orientation preferences along the (101) and (211) planes, which appeared at 2θ=36.09° and 2θ=54.33°, respectively.²³ The observed diffraction peaks at 2θ=25.3, 36.9, 37.9, 38.6, 48.0, 53.9, 55.1, and 62.1°, corresponded to anatase (101), (103), (004), (112), (200), (105), (221), and (213) planes, respectively. 18.24 The diffraction peak observed at 2θ=31° could be attributed to brookite (121).²⁵ Rutile has the smallest band gap of approximately 3.0 eV among the polymorphs, but it typically exhibits photocatalytic activity that is about an order of magnitude lower than that of anatase.³⁶ The optimal calcination condition was selected as 600° C., which preformed excellent atenolol degradation with pure anatase crystalline structure. FIG. 6B.

### Example 4—Effects of Lignin Content on Degradation Performance

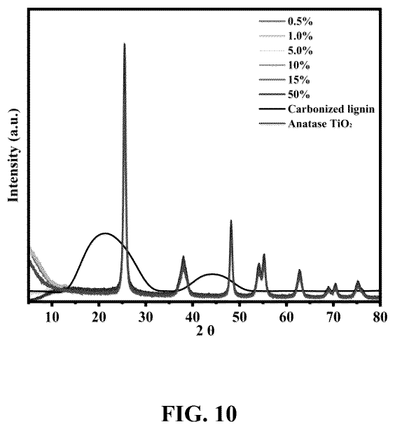

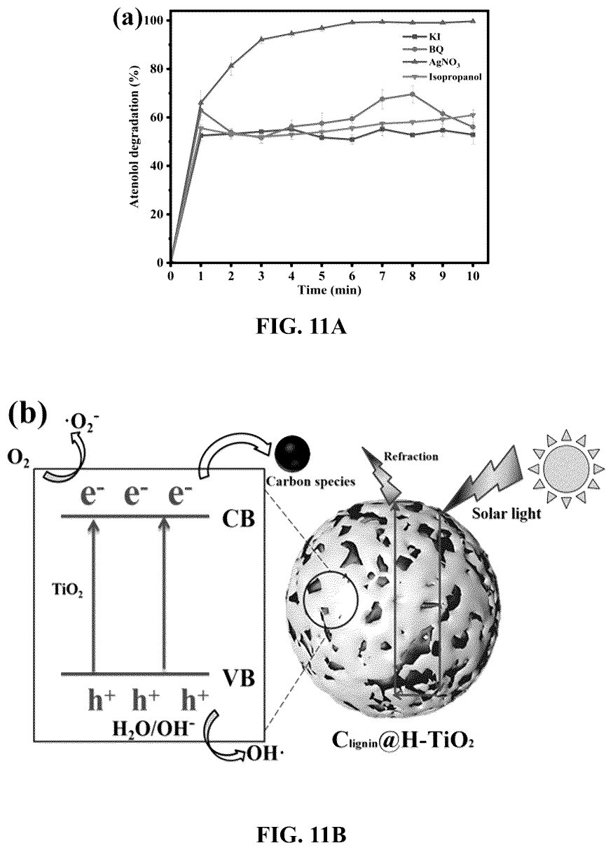

The lignin content also had a significant impact on the photocatalytic capacity. Lignin composition could influence the spheroidal morphology, reduce the TiO₂ band gap, and help match with the visible-light spectrum for light absorption.²⁷ As shown in FIG. 8, C_{lignin}@H—TiO₂ synthesized with 5.0% lignin showed a spherical shape with more and uniform holes. Additionally, as shown in FIG. 9, the incorporation of lignin into the TiO₂ increased the material conductivity, which could promote transferring the charges from the bulky TiO₂ structure to the oxidation reaction sites and facilitate photocatalysis. FIG. 7C shows the photocatalytic degradation of C_{lignin}@H—TiO₂ with varying amounts of lignin. In the ten-minute testing period, the degradation percentage increased as the amount of lignin increased from 0.5% to 5%. However, further increasing lignin content (i.e., 10% and 15%) had little improvement of the degradation efficiency. Consequently, C_{lignin}@H—TiO₂ with 5% lignin content was selected as the material composition for this study. The XRD measurements in FIG. 10 showed that all sample preparation formed the anatase phase TiO₂ after calcination at 600° C. for 90 minutes. Furthermore, the lignin content within 15% did not have any influence on the crystalline structures of C_{lignin}@H—TiO₂.

To investigate the effects of C_{lignin} content on the degradation performance, UV-vis and UV-vis DRS were conducted to analyze their visible light absorption and band gap. FIG. 7D. Anatase type TiO₂ sample exhibited an absorption edge near 400 nm, similar to that of the other samples with varying C_{lignin} content. The addition of C_{lignin} led to the red shifts in the light absorption in the visible region (>400 nm), which was also responsible of the gray/black color of the samples.²⁸ With the increasing C_{lignin} content, the absorption shifted to the longer wavelengths, particularly in the visible range correlating to the darker color of the materials. 29.30 This effect was more evident with higher C_{lignin} content, which led to much higher light absorption. The samples containing higher than 5% lignin showed black colors due to the carbon composite, which was responsible for the light absorption in the visible wavelength region. The samples were further characterized by UV-vis DRS with the reflectance mode to estimate the band gap values. FIG. 7E. With the increasing content of C_{lignin}, the C_{lignin}@H—TiO₂ photocatalysts exhibited lower band gap energies, except for the samples with more than 5% C_{lignin}, where the deep black color did not allow to estimate the band gap value of the titania phase.²⁸ The doped carbon is able to substitute the oxygen from the TiO₂ lattice and brings down the band gap by contributing its 2p orbitals.³¹ The band gap energy of TiO₂ was found to be about 3.2 eV and the band gap energy of C_{lignin}@H—TiO₂ with 5% C_{lignin} was found to be about 2.33 eV. FIG. 7E. A smaller band gap typically means that it is more capable of absorbing visible light and more effective in utilizing visible light for photocatalytic reactions. The results elucidate the reasons behind the influence of C_{lignin} on degradation performance, and indicate that C_{lignin}@H—TiO₂ with 5% C_{lignin} exhibits superior photocatalytic performance under visible light conditions.

### Example 5—Photocatalyst Stability

The photocatalysts stability is crucial for broad applications. FIG. 7F shows the atenolol degradation percentage after 10 minutes radiation upon ten consecutives cycles with C_{lignin}@H—TiO₂. The results suggested the catalyst degradation capacity remained about the same after 10 usage cycles, degrading 100% of the atenolol. The remarkable stability could be attributed to the uniform distribution of lignin within the crosslinking structures ensures a durable carbon modified TiO₂ framework, which efficiently separated photogenerated electrons and holes to sustain excited electrons and holes, introduced by the engineered C_{lignin} lattice structure.

### Example 6—Photocatalytic Degradation Mechanism

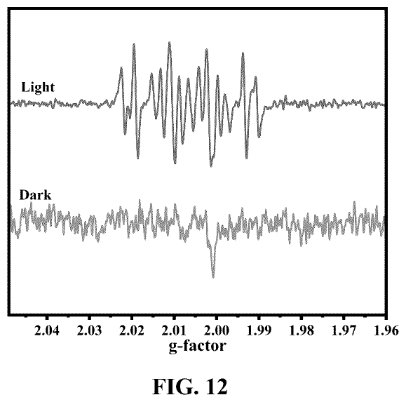

Selected scavengers were used to identify the primary reactive oxygen species. Benzoquinone (BQ), AgNO₃, isopropanol and potassium iodide (KI) were chosen as the capture agents of superoxide radicals (·O₂⁻), electron (e⁻), hydroxyl radicals (OH) and hole (h), respectively.^{32,33} The efficiency of photocatalytic degradation of atenolol by C_{lignin}@H—TiO₂ was reduced significantly after adding 0.5 mM of BQ, isopropanol and KI, individually. FIG. 11A indicates that ·O₂⁻, ·OH and h⁺ played key roles in photocatalytic degradation of atenolol. However, addition of 0.5 mM AgNO₃, did not significantly reduce the photocatalytic performance as about 100% degradation was achieved after 10 min, comparable to the catalysts' performance by itself. Since the AgNO₃ molecules scavenge e, the observation suggested that e might present in the C_{lignin}@H—TiO₂ photocatalytic degradation system at trace amounts, but not as the main photocatalytic active species. Apparently, the primary reactive oxygen species involved in the acetaminophen photodegradation include ·O₂⁻, ·OH radicals and h. ESR spin-trapping technique with DMPO is used to further verify the ·OH and ·O₂⁻ radicals.³⁴ As shown in FIG. 12, when irradiated with solar light, the ESR spectra of C_{lignin}@H—TiO₂ shows stronger signal than that of the dark control, which suggests the detection of ·OH and ·O₂⁻ radicals. Since the ·OH radicals are generally generated by holes,³⁵ the results are also strong evidence for the existence of h⁺.

The potential mechanism of the high photodegradation activity of C_{lignin}@H—TiO₂ nanocomposites is shown in FIG. 11B. The photocatalytic process included the photogenerated electron-hole separation and the free radical formation. The photocatalytic TiO₂ is well-known for the fast and easy recombination of photo-generated electron-hole pairs.^{36,37} Doping of C_{lignin} would have introduced lattice defects into TiO₂, which resulted in a narrower bandgap for visible light absorption. Under the visible lights, photons generated electrons and holes in C_{lignin} modified TiO₂. Furthermore, the available charge carriers could form more reactive species and facilitate photocatalysis. Besides, the separated electrons could react with the adsorbed O₂ to form ·O₂⁻ and catalyze the photo-oxidation reaction. The remaining holes in TiO₂ could also facilitate the redox reactions by forming ·OH radicals.⁴⁸ The hollow structure with potential sphere interior may give rise to surface reflectivity, which could further expand the light path within the C_{lignin}@H—TiO₂ and facilitate the light/matter interaction. As such, the photo-generated electrons could react with O₂ to form active oxidative species such as ·O₂⁻ and the holes react with H₂O to generate ·OH.³⁵

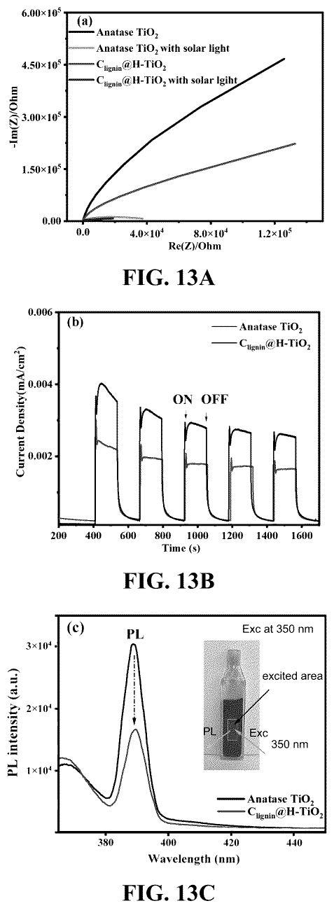

To further characterize the mechanism leading to the improved photocatalytic capacity, the photoluminescence emission, electrochemical impedance, and photocurrent response of C_{lignin}@H—TiO₂ were measured and compared to TiO₂. FIG. 13. The electrochemical impedance spectroscopy (EIS) is applied to characterize the charge transfer properties of samples, where Nyquist plots are presented in FIG. 13A. The C_{lignin}@H—TiO₂ exhibits a smaller arc radius than anatase TiO₂, indicating that C_{lignin}@H—TiO₂ is more efficient in transferring and separating photogenerated carriers.³⁹ The photocurrent is measured for C_{lignin}@H—TiO₂ and anatase TiO₂. FIG. 13B. The photocurrent density of C_{lignin}@H—TiO₂ is higher than that of anatase TiO₂, demonstrating that the separation rate of the photogenerated electrons and holes was enhanced.⁴⁰ From FIG. 13C, the decreased PL intensity of that C_{lignin}@H—TiO₂ compared to that of the anatase TiO₂ at 350 nm suggested a potential better charge separation and a complicated overall recombination process for the C_{lignin}@H—TiO₂ material.⁴¹

### Example 7—Degradation of Biological Molecules and Implication for Sterilization Applications

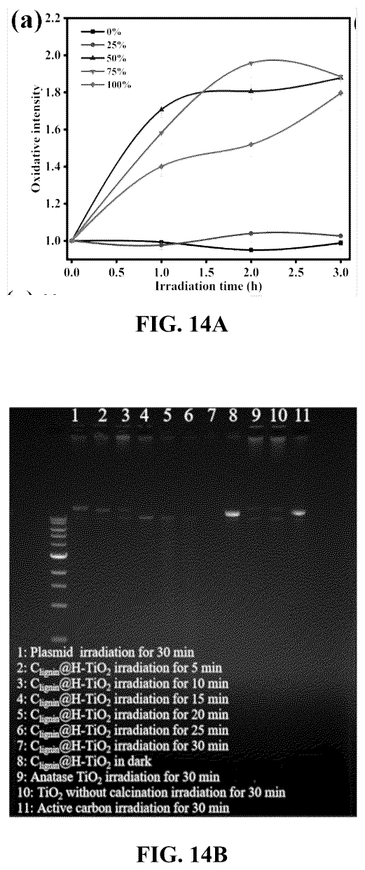

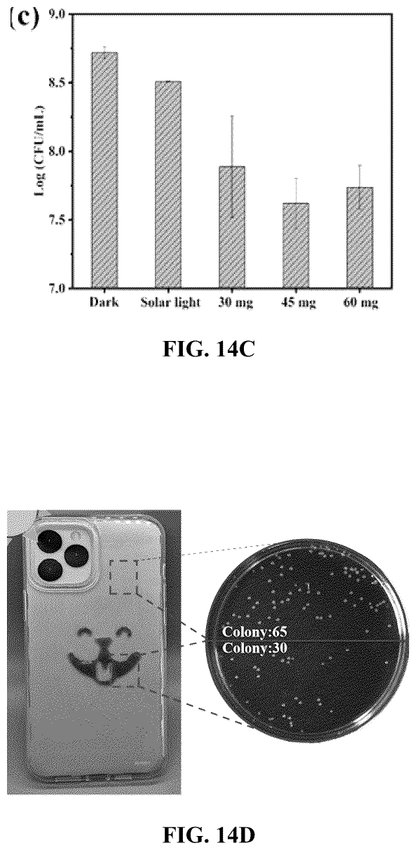

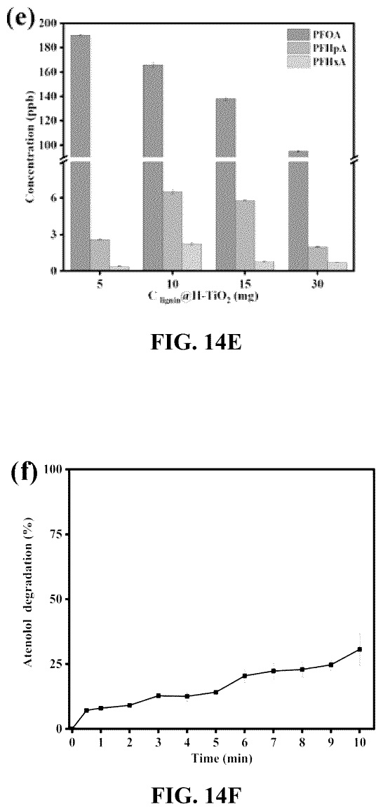

With the capacity of producing superoxide radicals (·O₂⁻) and hydroxyl radicals (.OH), the sterilization mechanism and examined cellular oxidative stress upon the photocatalyst treatment were further evaluated. The model bacteria used in this study was an environmental bacteria strain, Pseudomonas patida, which was a robust strain that could survive under extreme environmental conditions. The engineered strain has a fluorescence tag that produces florescence signal when the cell is under oxidative stress.⁴² The engineered Pseudomonas patida strain had a strong fluorescence signal when incubating with 0.5 mg of the photocatalyst. FIG. 14A. Furthermore, the cellular oxidative stress was clearly dependent on the light intensity. During the three-hour irradiation treatment, the cell experienced much stronger stress under the 50%, 75% and 100% light intensity. Interestingly, the cell population presented high fluorescence signal intensity under the 50% light intensity than the 100% light intensity, which could be attributed to a percentage of cells could have died from the strong oxidative stress, which prevented the gene expression and fluorescence signal production.

Possible mechanisms for cell damage were further reviewed and biological molecules were used as a surrogate to evaluate the molecular level oxidative damage. A plasmid of colibacillus origin with 11 k base pair was used to test the photocatalytic performance on large biological molecules. The plasmid molecule was also an ideal surrogate for DNA/RNA viruses in vivo. The plasmid was transferred and expressed in the E. coli HB101 competent cell to obtain 50 ng/uL for the degradation experiment. The purified plasmid was subject to C_{lignin}@H—TiO₂ treatment for five to 30 minutes and electro gel analysis. As shown in FIG. 14B, the plasmid was exposed to irradiation for different time periods. Lane one showed the intact plasmid that has not been treated with the photocatalysts. When the plasmid was mixed with the photocatalyst and gradually exposed to irradiation for five minutes, the plasmid began to degrade and produce small DNA fragments as shown in lane three. No more visible fragments were presented after 30 minutes of irradiation treatment (FIG. 14B lane 7). The C_{lignin}@H—TiO₂ catalysts performed much better than the anatase TiO₂ as shown in lane 9, where DNA fragments were visible after the 30 minutes irradiation. The plasmid/DNA degradation mechanism highlighted the potential application toward deactivating DNA/RNA viruses from the routine environment under ambient conditions.

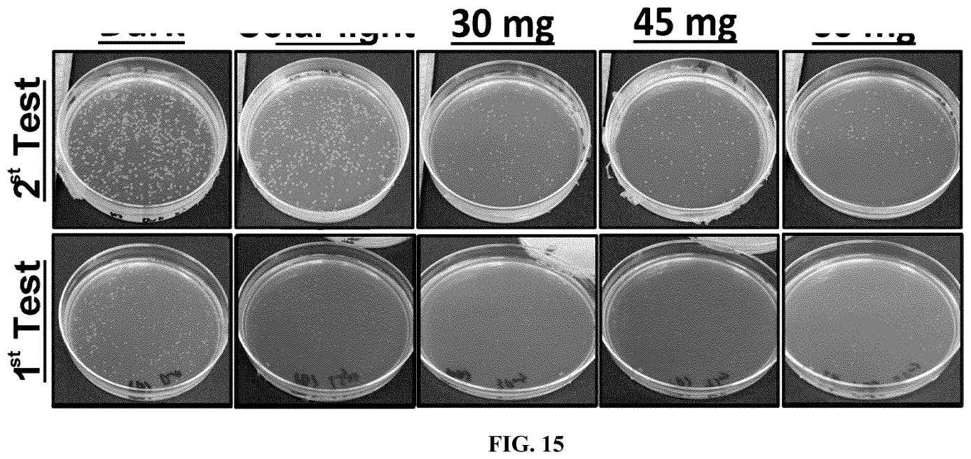

The antibacterial properties of C_{lignin}@H—TiO₂ were further evaluated by treating bacteria colonies (Pseudomonas putida A514) with the photocatalyst, as shown in FIG. 14C and FIG. 15. The initial concentration of the bacteria concentration was approximately 1×10⁹ CFU/mL, and the survival bacteria concentrations in different C_{lignin}@H—TiO₂ treatment groups were compared with the dark condition, and solar light exposure with one hour irradiation time. The bacterial concentration of the irradiated groups showed varying degrees of reduction. The 45 mg of photocatalyst presented the best sterilization effect, compared with that of the 30 mg and 60 mg materials. A log reduction of more than one suggested that more than 90% of the bacteria had been killed by the photocatalyst upon light irradiation. The cell death and damage mechanism would be more complex than the plasmid degradation. 43 However, the strong oxidative species produced by the photocatalyst could potentially lysis the cell, permeate the cell membrane and induce the molecular level degradation and intracellular damage.

### Example 8—Surface Coating for Sterilization

To further demonstrate the applicability of the photocatalyst in routine sterilization, C_{lignin}@H—TiO₂ was used in combination with other polymers for material surface coating. C_{lignin}@H—TiO₂ was incorporated into polyurethane resin and made a viscous solution, which was then applied to phone cases via screen printing.

TABLE 2
Sterilization performance of C_{lignin}@H—TiO₂ coating

| Samples | | Bacterial colonies/number |
|---|---|---|
| With | C_{lignin}@H—TiO₂ coated area | 48 |
| light | Disinfecting wipe wiped area | 49 |
| | Uncoated and unwiped areas | 82 |
| Without | C_{lignin}@H—TiO₂ coated area | 190 |
| light | Disinfecting wipe wiped area | 180 |
| | Uncoated and unwiped areas | 236 |

As displayed in FIG. 14D and Table 2, the C_{lignin}@H—TiO₂-coated areas showed a significantly lower number of bacterial colonies compared to the uncoated areas. Meanwhile, a commercial product (i.e., Clorox disinfecting wet wipes) was used as the control to demonstrate the photocatalyst effectiveness. The C_{lignin}@H—TiO₂-coated areas showed a significantly lower number of bacterial colonies compared to the uncoated areas and similar antibacterial effect compared to that of the Clorox disinfecting wet wipes wiped areas, indicating a remarkable antibacterial effect. Per fixed area, the bacteria colonies reduced by about 50% upon minutes of light irradiation. This highlighted the potential of C_{lignin}@H—TiO₂ for routine use for sterilizing material surfaces.

### Example 9—Chemical Degradation and Indication for Detoxification

The degradation capacity of C_{lignin}@H—TiO₂ on per and poly-fluoroalkyl substances (PFAS) was evaluated. PFOA (perfluorooctanoic acid), one of the so called stable “forever chemicals”, was selected to test the degradation efficiency. As such, C_{lignin}@H—TiO₂ was mixed with the PFOA solution and tested under different conditions. The degradation behaviors of different amounts of C_{lignin}@H—TiO₂ were shown in FIG. 14E. The PFOA removal rate increased with the increasing amount of C_{lignin}@H—TiO₂, and consequently, the degraded products were clearly observed by the LCMS measurements. In the three-hour testing time, 30 mg of the C_{lignin}@H—TiO₂ removed the most PFOA (˜50%) from the solution phase and produced degraded products of perfluoroheptanoic acid (PFHpA, C7) and perfluorohexanoic acid (PFHxA, C6). Interestingly, the disappearance of the PFOA molecule positively correlated with the catalyst quantity, which was not observed with the degraded products (C7 and C6). This could be due to the degradation kinetics, that the catalyst continuously degrades into the shorter chain degraded products. The degradation of perfluorooctanoic acid (PFOA) can potentially be initiated through two pathways: the hole oxidation pathway and the ·OH attack pathway.⁴⁴⁻⁴⁶ Both pathways involve the cleavage of the C—C bond between the carbon chain and carboxylic group, generating shorter chain perfluorocarboxylic acids (PFCAs) as intermediates.⁴⁷ The degradation process could begin at the terminal carboxylic end, where the h′ trapped e triggered formed the perfluorocarboxylate radicals (C7F15COO·). These unstable radicals initiated C—C bond cleavage between the carboxyl carbon and its adjacent carbon atoms, generating perfluorinated alkyl radicals that could react with ·OH to produce perfluorinated alcohol (C₇F₁₅OH). The alcohol would be further defluorinated to produce C₅F₁₃COF and C₆F₁₃COOH.⁴⁸ The intermediate radicals then could undergo successive loss of the terminal carboxylate group, addition of H₂O, elimination of HF, and hydrolysis, ultimately leading to the formation of CO₂ and F⁻.

Inspired by the antibacterial property when C_{lignin}@H—TiO₂ was mixed with polymers and coated on the device surface, the application of C_{lignin}@H—TiO₂ was further evaluated as a membrane material for chemical removal. A membrane composite by C_{lignin}@H—TiO₂ and polyurethane was fabricated to test atenolol removal. As shown in FIG. 6F, the membrane composed with C_{lignin}@H—TiO₂ achieved about 30% removal of atenolol after 10 minutes, which was lower than that of the C_{lignin}@H—TiO₂ itself (100% in 5 min, FIG. 7A). The reduction of photocatalytic degradation efficiency could be attributed to several factors, such as treatment on the membrane surface, rather than in the colloid solution; the mixture of the photocatalyst with polyurethane, which could reduce the active percentage of the material per unit area. Additionally, the polyurethane coating may also affect the reactivity of ·O₂⁻, e⁻, ·OH and h⁺, further contributing to the reduction of degradation efficiency. Nevertheless, given the short irradiation time (10 minutes) applied in this study, it is expected that for routine applications, the irradiation time would not be the constraint as the constant sunlight can catalyze chemical degradation continuously.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1. Makuła, P., Pacia, M., and Macyk, W. (2018). How to correctly determine the band gap energy of modified semiconductor photocatalysts based on UV-Vis spectra. J. Phys. Chem. Lett. 9, 6814-6817. https://doi.org/10.1021/acs.jpclett.8b02892
2. Hapeshi, E., Achilleos, A., Vasquez, M. I., Michael, C., Xekoukoulotakis, N. P., Mantzavinos, D., and Kassinos, D. (2010). Drugs degrading photocatalytically: kinetics and mechanisms of ofloxacin and atenolol removal on titania suspensions. Water Res. 44, 1737-1746. https://doi.org/10.1016/j.watres.2009.11.044
3. Ponkshe, A., and Thakur, P. (2022). Solar light-driven photocatalytic degradation and mineralization of beta blockers propranolol and atenolol by carbon dot/TiO₂ composite. Environ Sci. Pollut. Res. 29, 15614-15630. https://doi.org/10.1007/s11356-021-16796-w
4. Sarkar, S., Chakraborty, S., and Bhattacharjee, C. (2015). Photocatalytic degradation of pharmaceutical wastes by alginate supported TiO₂ nanoparticles in packed bed photo reactor (PBPR). Ecotoxicol. Environ. Saf. 121, 263-270. https://doi.org/10.1016/j.ecoenv.2015.02.035
5. Radjenović, J., Sirtori, C., Petrović, M., Barceló, D., and Malato, S. (2009). Solar photocatalytic degradation of persistent pharmaceuticals at pilot-scale: kinetics and characterization of major intermediate products. Appl. Catal., B 89, 255-264. https://doi.org/10.1016/j.apcatb.2009.02.013
6. Ioannou, L., Hapeshi, E., Vasquez, M., Mantzavinos, D., and Fatta-Kassinos, D. (2011). Solar/TiO2 photocatalytic decomposition of β-blockers atenolol and propranolol in water and wastewater. Sol. Energy 85, 1915-1926. https://doi.org/10.1016/j.solener.2011.04.031
7. Bhatia, V., Malekshoar, G., Dhir, A., and Ray, A. K. (2017). Enhanced photocatalytic degradation of atenolol using graphene TiO₂ composite. J. Photochem. Photobiol., A 332, 182-187. https://doi.org/10.1016/j.jphotochem.2016.08.029
8. Liu, D., Chen, Y., Guan, R., Zhao, J., Jin, H., Zhang, S., and Shang, Q. (2022). Photocatalytic performance of heterojunction S-Tyr-NDI-Tyr/TiO₂ formed by self-assembled naphthalimide derivatives and titanium dioxide. Chemosphere 296, 134046. https://doi.org/10.1016/j.chemosphere.2022.134046
9. Ji, Y., Zhou, L., Ferronato, C., Yang, X., Salvador, A., Zeng, C., and Chovelon, J.-M. (2013). Photocatalytic degradation of atenolol in aqueous titanium dioxide suspensions: kinetics, intermediates and degradation pathways. J. Photochem. Photobiol., A 254, 35-44. https://doi.org/10.1016/j.jphotochem.2013.01.003
10. Marquez, G., Rodriguez, E. M., Maldonado, M. I., and Alvarez, P. M. (2014). Integration of ozone and solar TiO₂-photocatalytic oxidation for the degradation of selected pharmaceutical compounds in water and wastewater. Sep. Purif. Technol. 136, 18-26.
11. Chen, M., Wu, L., Zhou, S., and You, B. (2006). A method for the fabrication of monodisperse hollow silica spheres. Adv. Mater. 18, 801-806. https://doi.org/10.1002/adma.200501528
12. Djellabi, R., Yang, B., Xiao, K., Gong, Y., Cao, D., Sharif, H.M.A., Zhao, X., Zhu, C., and Zhang, J. (2019). Unravelling the mechanistic role of TiOC bonding bridge at titania/lignocellulosic biomass interface for Cr (VI) photoreduction under visible light. J. Colloid Interface Sci. 553, 409-417. https://doi.org/10.1016/j.jcis.2019.06.052
13. An, G., Ma, W., Sun, Z., Liu, Z., Han, B., Miao, S., Miao, Z., and Ding, K. (2007). Preparation of titania/carbon nanotube composites using supercritical ethanol and their photocatalytic activity for phenol degradation under visible light irradiation. Carbon 45, 1795-1801. https://doi.org/10.1016/j.carbon.2007.04.034
14. Wu, W., Liu, T., Deng, X., Sun, Q., Cao, X., Feng, Y., Wang, B., Roy, V.A., and Li, R. K. (2019). Ecofriendly UV-protective films based on poly(propylene carbonate) biocomposites filled with TiO₂ decorated lignin. Int. J. Biol. Macromol. 126, 1030-1036. https://doi.org/10.1016/j.ijbiomac.2018.12.273
15. Kumar, R., Singh, B. K., Soam, A., Parida, S., Sahajwalla, V., and Bhargava, P. (2020). In situ carbon-supported titanium dioxide (ICS-TiO₂) as an electrode material for high performance supercapacitors. Nanoscale Adv. 2, 2376-2386. https://doi.org/10.1039/DONA00014K
16. Zheng, X., Gao, M., Liang, C., Wang, S., and Wang, X. (2022). Expanded graphite supported TiO₂ composites using polyaniline as the anchor: Improved catalytic performance for the electro-Fenton-like reaction. Electrochim. Acta 428, 140910. https://doi.org/10.1016/j.electacta.2022.140910
17. Srisasiwimon, N., Chuangchote, S., Laosiripojana, N., and Sagawa, T. (2018). TiO₂/lignin-based carbon composited photocatalysts for enhanced photocatalytic conversion of lignin to high value chemicals. ACS Sustainable Chem. Eng. 6, 13968-13976. https://doi.org/10.1021/acssuschemeng.8b02353
18. Li, Y., Shen, Q., Guan, R., Xue, J., Liu, X., Jia, H., Xu, B., and Wu, Y. (2020). AC@TiO₂ yolk-shell heterostructure for synchronous photothermal-photocatalytic degradation of organic pollutants. J. Mater. Chem. C 8, 1025-1040. https://doi.org/10.1039/C9TC05504E
19. Gan, L.-Y., Zhang, Q., Guo, C.-S., Schwingenschlogl, U., and Zhao, Y. (2016). Two-dimensional MnO2/graphene interface: half-metallicity and quantum anomalous hall state. J. Phys. Chem. C 120, 2119-2125. https://doi.org/10.1021/acs.jpcc.5b08272
20. Zhang, W., Ji, X., Zeng, C., Chen, K., Yin, Y., and Wang, C. (2017). A new approach for the preparation of durable and reversible color changing polyester fabrics using thermochromic leuco dye-loaded silica nanocapsules. J. Mater. Chem. C 5, 8169-8178. https://doi.org/10.1039/C7TC02077E
21. Zhang, Z., Xiong, Z., Zhao, C., Guo, P., Wang, H., and Gao, Y. (2021). In-situ carbon-coated TiO₂ boosting the visible-light photocatalytic hydrogen evolution. Appl. Surf. Sci. 565, 150554. https://doi.org/10.1016/j.apsusc.2021.150554
22. Li, M., Qiu, J., Xu, J., and Yao, J. (2020). Cellulose/TiO₂-based carbonaceous composite film and aerogel for highly efficient photocatalysis under visible light. Ind. Eng. Chem. Res. 59, 13997-14003. https://doi.org/10.1021/acs.iecr.0c01682
23. Chen, G., Chen, J., Song, Z., Srinivasakannan, C., and Peng, J. (2014). A new highly efficient method for the synthesis of rutile TiO₂. J. Alloys Compd. 585, 75-77. https://doi.org/10.1016/j.jallcom.2013.09.056
24. Zarattini, M., Dun, C., Isherwood, L. H., Felten, A., Filippi, J., Gordon, M.P., Zhang, L., Kassem, O., Song, X., and Zhang, W. (2022). Synthesis of 2D anatase TiO₂ with highly reactive facets by fluorine-free topochemical conversion of 1T-TiS2 nanosheets. J. Mater. Chem. A 10, 13884-13894. https://doi.org/10.1039/DITA06695A
25. Solati, E., Aghazadeh, Z., and Dorranian, D. (2020). Effects of liquid ablation environment on the characteristics of TiO₂ nanoparticles. J. Clust. Sci. 31, 961-969. https://doi.org/10.1007/s10876-019-01701-w
26. Odling, G., and Robertson, N. (2015). Why is anatase a better photocatalyst than rutile? The importance of free hydroxyl radicals. ChemSusChem 8, 1838-1840. https://doi.org/10.1002/cssc.201500298
27. Shayegan, Z., Haghighat, F., and Lee, C.-S. (2020). Carbon-doped TiO₂ film to enhance visible and UV light photocatalytic degradation of indoor environment volatile organic compounds. Journal of Environmental Chemical Engineering 8, 104162. https://doi.org/10.1016/j.jece.2020.104162
28. Gómez-Avilés, A., Peñas-Garzón, M., Bedia, J., Rodriguez, J., and Belver (2019). C-modified TiO₂ using lignin as carbon precursor for the solar photocatalytic degradation of acetaminophen. Chem. Eng. J 358, 1574-1582. https://doi.org/10.1016/j.cej.2018.10.154
29. Chen, K., Zhou, X., Wang, D., Li, J., and Qi, D. (2022). Synthesis and characterization of a broad-spectrum TiO₂@ lignin UV-protection agent with high antioxidant and emulsifying activity. Int. J. Biol. Macromol. 218, 33-43. https://doi.org/10.1016/j.ijbiomac.2022.06.190
30. Chen, X., Sun, H., Zhang, J., Zelekew, O.A., Lu, D., Kuo, D.-H., and Lin, J. (2019). Synthesis of visible light responsive iodine-doped mesoporous TiO₂ by using biological renewable lignin as template for degradation of toxic organic pollutants. Appl. Catal., B 252, 152-163. https://doi.org/10.1016/j.apcatb.2019.04.034
31. Negi, C., Kandwal, P., Rawat, J., Sharma, M., Sharma, H., Dalapati, G., and Dwivedi, C. (2021). Carbon-doped titanium dioxide nanoparticles for visible light driven photocatalytic activity. Appl. Surf. Sci 554, 149553. https://doi.org/10.1016/j.apsusc.2021.149553
32. Wang, Y., Lin, L., Dong, Y., and Liu, X. (2022). Facile synthesis of MOF-808/AgI Z-scheme heterojunction with improved photocatalytic performance for the degradation of tetracycline hydrochloride under simulated sunlight. New J. Chem. 46, 16584-16592. https://doi.org/10.1039/D2NJ03301A
33. Wen, Y., Rentería-Gomez, A. n., Day, G.S., Smith, M. F., Yan, T.-H., Ozdemir, R. O. K., Gutierrez, O., Sharma, V. K., Ma, X., and Zhou, H.-C. (2022). Integrated photocatalytic reduction and oxidation of perfluorooctanoic acid by metal-organic frameworks: key insights into the degradation mechanisms. J. Am. Chem. Soc. 144, 11840-11850. https://doi.org/10.1021/jacs.2c04341
34. Zhu, Y., Wang, Y., Chen, Z., Qin, L., Yang, L., Zhu, L., Tang, P., Gao, T., Huang, Y., and Sha, Z. (2015). Visible light induced photocatalysis on CdS quantum dots decorated TiO₂ nanotube arrays. Appl. Catal., A 498, 159-166. https://doi.org/10.1016/j.apcata.2015.03.035
35. Hashimoto, K., Irie, H., and Fujishima, A. (2005). TiO₂ photocatalysis: a historical overview and future Jpn. J. Appl. prospects. Phys. 44, 8269. https://doi.org/10.1143/JJAP.44.8269
36. Yu, J., Ma, T., and Liu, S. (2011). Enhanced photocatalytic activity of mesoporous TiO₂ aggregates by embedding carbon nanotubes as electron-transfer channel. Phys. Chem. Chem. Phys. 13, 3491-3501. https://doi.org/10.1039/C0CP01139H
37. Wang, C., Cao, M., Wang, P., Ao, Y., Hou, J., and Qian, J. (2014). Preparation of graphene-carbon nanotube-TiO₂ composites with enhanced photocatalytic activity for the removal of dye and Cr (VI). Appl. Catal., A 473, 83-89. https://doi.org/10.1016/j.apcata.2013.12.028
38. Huang, Y., Kang, S., Yang, Y., Qin, H., Ni, Z., Yang, S., and Li, X. (2016). Facile synthesis of Bi/BizWO6 nanocomposite with enhanced photocatalytic activity under visible light. Appl. Catal., B 196, 89-99. https://doi.org/10.1016/j.apcatb.2016.05.022
39. Liang, Z., Bai, X., Hao, P., Guo, Y., Xue, Y., Tian, J., and Cui, H. (2019). Full solar spectrum photocatalytic oxygen evolution by carbon-coated TiO₂ hierarchical nanotubes. Appl. Catal., B 243, 711-720. https://doi.org/10.1016/j.apcatb.2018.11.017
40. Jiao, W., Zhang, L., Yang, R., Ning, J., Xiao, L., Liu, Y., Ma, J., Mahmood, N., and Jian, X. (2022). Synthesis of monolayer carbon-coated TiO₂ as visible-light-responsive photocatalysts. Appl. Mater. Today 27, 101498. https://doi.org/10.1016/j.apmt.2022.101498
41. Velmurugan, R., Krishnakumar, B., Subash, B., and Swaminathan, M. (2013). Preparation and characterization of carbon nanoparticles loaded TiO₂ and its catalytic activity driven by natural sunlight. Sol. Energy Mater. Sol. Cells 108, 205-212. https://doi.org/10.1016/j.solmat.2012.09.018
42. Jayakody, L. N., Johnson, C. W., Whitham, J. M., Giannone, R. J., Black, B. A., Cleveland, N. S., Klingeman, D. M., Michener, W. E., Olstad, J. L., and Vardon, D. R. (2018). Thermochemical wastewater valorization via enhanced microbial toxicity tolerance. Energy Environ. Sci. 11, 1625-1638. https://doi.org/10.1039/C8EE00460A
43. Adhikari, S., Banerjee, A., Eswar, N.K., Sarkar, D., and Madras, G. (2015). Photocatalytic inactivation of E. Coli by ZnO—Ag nanoparticles under solar radiation. RSC Adv. 5, 51067-51077. https://doi.org/10.1039/C5RA06406F
44. Song, Z., Dong, X., Wang, N., Zhu, L., Luo, Z., Fang, J., and Xiong, C. (2017). Efficient photocatalytic defluorination of perfluorooctanoic acid over BiOCI nanosheets via a hole direct oxidation mechanism. Chem. Eng. J. 317, 925-934. https://doi.org/10.1016/j.cej.2017.02.126
45. Wang, N., Lv, H., Zhou, Y., Zhu, L., Hu, Y., Majima, T., and Tang, H. (2019). Complete defluorination and mineralization of perfluorooctanoic acid by a mechanochemical method using alumina and persulfate. Environ. Sci. Technol. 53, 8302-8313. https://doi.org/10.1021/acs.est.9b00486
46. Shen, Y., Zhu, C., Song, S., Zeng, T., Li, L., and Cai, Z. (2019). Defect-abundant covalent triazine frameworks as sunlight-driven self-cleaning adsorbents for volatile aromatic pollutants in water. Environ. Sci. Technol. 53, 9091-9101. https://doi.org/10.1021/acs.est.9b02222
47. Song, Z., Dong, X., Fang, J., Xiong, C., Wang, N., and Tang, X. (2019). Improved photocatalytic degradation of perfluorooctanoic acid on oxygen vacancies-tunable bismuth oxychloride nanosheets prepared by a facile hydrolysis. J. Hazard. Mater. 377, 371-380. https://doi.org/10.1016/j.jhazmat.2019.05.084
48. Tang, H., Xiang, Q., Lei, M., Yan, J., Zhu, L., and Zou, J. (2012). Efficient degradation of perfluorooctanoic acid by UV-Fenton process. Chem. Eng. J. 184, 156-162. https://doi.org/10.1016/j.cej.2012.01.020

We claim:

1. A photocatalyst composition comprising a metal oxide semiconductor modified with a carbon dopant derived from a carbon precursor material, wherein the photocatalyst composition comprises a hollow, nanoparticle structure having a spherical or spheroid shape, and wherein the photocatalyst composition is capable of facilitating remediation of a contaminant upon irradiation via light within the visible light range.

2. The photocatalyst composition of claim 1, wherein the metal oxide semiconductor is ZnO, TiO₂, NiO, Ni₂O₃, CuO, Cu₂O, Co₂O₃, W₂O₃, WO₃, V₂O₅, Fe₃O₄, FeO, FeO₂, Fe₂O₃, RuO₂, RuO₄, Re₂O₇, MoO₃, ZrO₂, Cr₂O₃, CrO, CrO₂, SnO₂, CeO₂, FeS₂, CdS, NiS, NiS₂, Ni₃S₂, Ni₃S₄, CoS, Bi₂S₃, ZnS, CuS, Cu₂S, PbS, MoS₂, and/or WS₂.

3. The photocatalyst composition of claim 2, wherein the metal oxide semiconductor is TiO₂.

4. The photocatalyst composition of claim 1, wherein the carbon precursor material is lignin, chitosan, chitin, cellulose, guar, alginic acid, hemicellulose, dextran, xanthan, agar, starch, sucrose, dextrose, silk, wool, DNA, RNA, protein, glycogen, amylose, enzymes, polypeptides, pectin, keratin, collagen, hyaluronic acid or their derivatives and renewable synthetic polymers comprise of polylactic acid, polyglycolic acid, polycaprolactone, polyaspartic acid, polyphosphazenes, polyhydroxyalkanoate, polybutyrate, poly-3-hydroxybutyrate, poly(lactide-co-glycolide), poly(hydroxybutyrate-co-hydroxyvalerate), poly(butylene succinate), poly(butylene succinate adipate), poly(ethylene succinate), or a combination thereof.

5. The photocatalyst composition of claim 4, wherein the carbon precursor material is lignin.

6. The photocatalyst composition of claim 5, comprising 5% to 15% lignin by weight.

7. The photocatalyst composition of claim 1, wherein the metal oxide semiconductor it TiO₂ and the carbon precursor material is lignin, and wherein the photocatalyst composition is formed by mixing titanium (IV) n-butoxide with a dispersion of lignin nanoparticles; subjecting the titanium (IV) n-butoxide to hydrolysis, thereby copolymerizing the titanium (IV) n-butoxide with the lignin nanoparticles; and calcining the resulting copolymer, thereby establishing a lattice of Ti—O—C and Ti—O—Ti bonds.

8. The photocatalyst composition of claim 1, having an average nanoparticle diameter of about 250 to 600 nm.

9. The photocatalyst composition of claim 1, having a band gap of 1.77 eV to 2.33 eV.

10. The photocatalyst composition of claim 1, further comprising a substrate fixed with the nanoparticle structure, wherein the substrate is a filter comprising porous PTFE, non-woven or woven textile, glass/quartz wool, cellulose, plastics, polymers, resins, metal, ceramic, activated or porous carbon, or zeolites.

11. The photocatalyst composition of claim 1, further comprising a substrate fixed with the nanoparticle structure, wherein the substrate is a membrane comprising polyurethane.

12. The photocatalyst composition of claim 1, further comprising a substrate fixed with the nanoparticle structure, wherein the substrate is a cleaning wipe.

13. The photocatalyst composition of claim 1, further comprising additives such as carriers, propellants, adhesives, plasticizers, pigments, colorants, glidants, stabilization agents, pore formers and surfactants.

14. The photocatalyst composition of claim 13, comprising an aqueous carrier.

15. A method for remediating a contaminant at a site, the method comprising contacting an effective amount of the photocatalyst composition according to claim 1 with the site and irradiating the photocatalyst with electromagnetic radiation.

16. The method of claim 15, wherein the electromagnetic radiation has a wavelength of 10 nm to 860 nm.

17. The method of claim 15, wherein the electromagnetic radiation has a wavelength of 380 nm to 820 nm.

18. The method of claim 15, wherein the photocatalyst composition is formulated as a dispersion, a colloid, an aqueous solution or an aerosol, and wherein the photocatalyst is contacted with the contaminated site via spreading, spraying or pouring.

19. The method of claim 15, wherein the photocatalyst composition is fixed to a substrate that contacts the contaminated site, wherein the substrate is a cleaning wipe, a matrix, a membrane or a filter.

20. The method of claim 15, wherein the contaminated site is a solid, liquid, gas, plasma or combination thereof.
